(19) United States
(12) Patent Application Publication
(10) Pub. No.: US 2025/0288772 A1
(43) Pub. Date: Sep. 18, 2025

(54) Pod Chamber For Immersing A User In A Multisensory Experience Delivered In Concert With An Audio Beat Frequency

(71) Applicant: Recharge Pod Limited, Nicosia (CY)

(72) Inventors: Tavis G. Keen, Gianyar (ID); I Wayan Dani Pranata, Denpasar Timur (ID)

(21) Appl. No.: 19/074,897

(22) Filed: Mar. 10, 2025

Related U.S. Application Data
(60) Provisional application No. 63/564,430, filed on Mar. 12, 2024.

(51) Int. Cl. A61M 21/00; A61M 21/02; G06F 3/01; H04R 1/02; H04R 3/12; H04R 5/02; H04S 3/00

(52) U.S. Cl. CPC A61M 21/0094; A61M 21/02; G06F 3/016; H04R 1/028; H04R 3/12; H04R 5/02; H04S 3/008; A61M 2021/0016; A61M 2021/0022; A61M 2021/0027; A61M 2021/005; A61M 2021/0055; A61M 2210/086; A61M 2210/1003; H04R 2499/15; H04S 2400/01

(57) ABSTRACT

Techniques are disclosed for a pod chamber for immersing a user in a multisensory experience delivered in concert with an audio beat frequency generated by a first set of audio speakers positioned on each side of the user's head. A second set of audio speakers delivers an audio performance in concert with the audio beat frequency. A stroboscopic light source provides a strobing pattern of light also in concert with the audio beat frequency as well as with the audio performance. Additional input from tactile transducers is also delivered in concert with the audio beat frequency and the audio performance. Still other elements, such as pulsed electromagnetic field generator, olfactory aromatic diffuser and screens with visuals are provided in the pod chamber to achieve immersion in the multisensory experience and more specifically to induce in the user a sense of participation in the multisensory experience.

### CROSS-REFERENCE TO RELATED APPLICATIONS

This application claims priority from U.S. Provisional Patent Application No. 63/564, 430 filed on Mar. 12, 2024 and which is incorporated by reference herein for all purposes in its entirety.

### FIELD OF THE INVENTION

The present invention relates generally to methods and apparatus that provide multisensory experiences and more specifically to a pod chamber that immerses a user in a multisensory experience comprising various stimuli.

### BACKGROUND ART

To date, there have been systems for providing sensory stimulation for enhanced entertainment as well as for therapy, stress management and improved body functioning. Existing concepts of sensory stimulation have dealt with creating enhanced visual system displays, audio variations and/or olfactory inputs to create different moods for one or more persons subjected to such systems.

In the entertainment industry, basic visual systems have been modified to create three-dimensional (3D) imagining to make visual presentations more realistic. Other variations of image projection include creating multiple lighting and strobing effects of image presentation for stimulating our mind's responses to different colors, shades, light intensities and sequences.

As with changes in visual stimuli, changes in audio stimuli can also have an effect on an individual's feelings, mood and health. Dynamic audio presentations tend to increase heart rate and biorhythm functioning whereas more melodic softer or quieter audio presentations tend to create a soothing effect on individuals.

By combining different audio programming with visual presentations, the movie industry has attempted to change the manner in which people become involved while viewing a film. In some prior art presentations, it has been proposed to provide aromatic essences which are released into a theater to combine with the visual and audio presentations to achieve yet a higher degree of individual involvement with the presentation of a film.

Another stimulation includes providing movement or vibration of seats in theaters to stimulate the individual through a sense of touch or through a sense of bodily orientation which changes as the visual and audio presentations change. By combining numerous sensory stimuli, a heightened interaction can be established between individuals and a programmed presentation.

U.S. Patent Publication 2020/0246579 A1 to Cohen describes an apparatus capable of creating synchronized sound, vibration and magnetic field stimulation, for the purpose of habituating and inhibiting brain function while stimulating the human spiritual energy system. The apparatus comprises an amplifier and transducers built into a comfortable seating arrangement including a support structure and a motion platform. The support structure, such as a chair, rests upon the motion platform, which is adapted to impart three-dimensional motion to the support structure. The apparatus uses layered music to create synchronized sounds, vibrations and magnetic fields.

US Patent Publication 2016/0144150 A1 to Berg teaches a system and method utilizing synchronized colors, sounds, and aroma therapy. The visual, auditory, and aromatic stimulation is intended to alleviate stress and/or anxiety by relaxing and stimulating the brain, thereby correcting any neurophysical imbalances. Reduction in the levels of stress and/or anxiety is achieved through the use of the auditory, visual, and aromatic sequences which are individually specific and systematic. The system and method are preferably provided in multiple sessions with each session providing both a mechanism for providing treatment as well as assessment, which is used to form the basis for future treatments.

US Patent Publication No. 2015/0150745 A1 to Strlek teaches a set-down device for setting down on a neurophysiological stimulator for transmitting movements generated by the system.

The device comprises a set down side for setting down on the stimulator,, and a transmission side for transmitting the movements received via the set-down side, wherein the transmission side has a partially projecting stimulation element, which is suitable for transmitting the movements for neurophysiological stimulation. The device stimulates an object and includes a movement unit and a drive unit for generating the movement, the drive unit being coupled to the movement unit, wherein the drive unit is designed as a tumbling and/or spinning unit and generates a tumbling and/or spinning movement.

US Patent Publication No. 2010/0130812 A1 to Martel discloses Light modulation devices. At least one Low Frequency Oscillator (LFO) is used to create an oscillating signal for driving an intensity parameter, a color parameter or both in a light modulator. The oscillating signal may be mixed with a base signal. The modulated signal driving either of the intensity or color parameters may be simple or complex. Systems having a plurality of light projection devices, each associated with a corresponding light modulation device, are also provided. Such Light modulation systems may be used for a variety of applications.

US Patent Publication No. 2008/0125620 A1 to McNew teaches a system, apparatus, and method for therapeutic light and sound. The method comprises the steps of exposing a user to spectral light and laterally directed sound and adjusting the manner of exposure sufficient to elicit a desired autonomic nervous response.

U.S. Pat. No. 11,779,227 B2 to Bartosch describes a multisensory environment apparatus for providing stress reduction to a user. The apparatus includes a support frame that defines a recess. signal generators, somatosensory signal modules, Auditory olfactive signal modules, visual signal modules, and vestibular signal modules are positioned to deliver the associated signals to a user positioned in the recess. The delivery of the auditory and vibrotactile signals to the user work to meet the user's vital functions where they are, entrain them, and guide them to a more relaxed state, helping to reduce stress. The combination of the signals and the apparatus provide short term relief of acute stress, and long-term benefits by breaking the cycle of chronic stress.

U.S. Pat. No. 10,737,054 B1 to Lynn discloses a system including a chamber structure having a substantially oval or “egg” shaped outer shell. The system may also include an interior formed within the outer shell. A user area may be provided in the chamber structure substantially centered front to back and side to side within the outer shell. The system may also include at least one sound emitting device positioned to provide at least one person in the user area with a prescribed sound therapy. The system may also include at least one light emitting device selected to provide the person in the user area with a prescribed light therapy.

U.S. Pat. No. 10,500,369 B2 to Kuhl teaches a synergistic system and protocol for stress management and reduction in a person in need thereof. In particular, an improved system and method for relaxing, reducing stress in, and improving the well-being of an individual is disclosed such that after treatment or use of the system the individual can recall or come back to a decreased stress state obtained during treatment, thereby reducing their present stress level. It is an objective of their system to provide a non-pharmaceutical method and system of treatment that merges several stress reduction methods into an effective treatment protocol that can be used in a variety of environments and on any age of individual as a means to reduce anxiety and stress.

U.S. Pat. No. 6,702,767 B1 to Douglas describes a multisensory stimulation system and method of use. The system includes a housing articulate on a support so as to align a marked viewport with an individual. The housing defines a viewing chamber which is an extension of a reflective light and image optical system chamber. The chamber receives light and images from one or more video monitors or images projectors and from alternate sources such as black lights and strobe light devices. The system also includes an aromatic sensory component, tactile sensation devices, an audio input system, and audio delivery devices.

There are also various systems available in the industry that attempt to provide multisensory stimulation environments to human subjects/patients for the achievement of various objectives. These include the pod from Biohacking Orb (https://bichackingorb.com/), wellness technology from Rebalance (https://www.rebalancetech.com/), products from Harmonic Egg (https://harmonicegg.com/) and the therapeutic device offered by Life Vessel (https://lifevessel.com/).

Other devices currently in the market are:
Ajna Light https://ajnalight.com/
Pandora Star https://pandorastar.co.uk/
Lucia Light https://lucialightexperience.com/
Enkidu Light https://www.enkidulight.com/
Roxiva https://roxiva.com/

The main problem with all of these lights is their inability to easily adjust the intensity during the sessions. Further, the abovementioned stroboscopic lights operate as stand-alone devices. When people are starting to shift into altered states of consciousness, sometimes they become aware of their own physical selves, movements or even their breath. Often the user is self-conscious and would not allow themselves to really drop into a deeper state, because their eyes are closed and they are mindful of other people: (a) watching them, (b) hearing them if they make noises, or (c) if they twitch or move as they shift into different states.

Furthermore, despite the prevalence of prior art, there has not been a system that provides a multisensory experience to a user that is in concert with an audio beat frequency. Most multifaceted sensory stimulation systems have been provided in the entertainment industry for purposes of stimulating the senses of an audience. Such systems are not only costly and complex, but they are also not adequate for use with specific individuals.

Thus, there is a need of a multisensory stimulation system that can be varied by the user based on their response levels to the stimulation and their personal needs. Such a multisensory stimulation system would be used not only for purposes of entertainment but more importantly, for purposes of mental and physical therapy in order to reduce stress, and to reinvigorate the mind and body for undertaking strenuous takes afterwards. The present invention addresses these deficiencies.

### OBJECTS OF THE INVENTION

In view of the shortcomings of the prior art, it is an object of the invention to provide a pod chamber in which an immersive multisensory experience is delivered to the user that induces a sense of participation by the user in the multisensory experience.

It is also an object of the invention to include in the multisensory experience, stroboscopic lights and vibrations in concert with a binaural beat and an audio performance.

It is also an object of the invention to include low-frequency pulsed electromagnetic waves in the multisensory experience delivered to the user.

It is also an object of the invention to produce the binaural beat at 40 Hertz, as well as delivering light, vibration and pulsed electromagnetic frequency stimuli at this frequency.

It is further an object of the invention to deliver visuals to the user during the multisensory experience in concert with the binaural beat and the audio performance.

It is also an object of the invention to use stroboscopic lights for inducing a “mind's eye” imagery leading to a hypnagogic effect in the user.

It is also an object of the invention to display conventional visual content on custom-integrated roof screens of the pod.

It is further an object of the invention to include aromas in concert with the binaural beat and the audio performance.

Still other objects and advantages of the invention will become apparent upon reading the summary and the detailed description in conjunction with the drawing figures.

### SUMMARY OF THE INVENTION

A number of objects and advantages of the invention are achieved by apparatus and methods for a pod chamber in which a user is reclined on a chair. There is a first set of audio speakers behind the head of the user reclined in the chair for delivering two coherent sound waves that produce an audio beat frequency. This audio beat frequency is referred to as binaural beat and is an auditory illusion or a “phantom” beat perceived by the brain and has a frequency equal to the difference between the frequencies of the two coherent sound waves or tones.

There is also a second set of audio speakers that deliver an audio performance to the user reclined in the chair in the pod chamber. The audio performance may be a music or audio track or any suitable musical audio recording. The audio performance is preferably delivered to the user in concert with the audio beat frequency produced as a result of the coherent sounds waves generated from the first set of speakers.

To be considered in concert with the audio beat frequency, audio performance incorporates the audio beat frequency as an integral part. Among other techniques, one way to accomplish this is by including a base line in audio performance that incorporates the audio beat frequency. The incorporation may be direct i.e. a direct part of the base line, or indirect i.e. not expressly included in the base line but relied upon for completing the base line.

There is preferably a stroboscopic light or lamp also that delivers a strobing pattern to the user. In the preferred embodiment, the strobing pattern is in concert with the audio beat frequency as well as the audio performance. This induces rapid eye movement (REM) in the user if the user's eyes are closed. There is preferably a set of vibro-tactile transducers also that deliver vibrations to suitable points of the body of the user. Once again, the vibrations are preferably delivered to the user in concert with the audio beat frequency and the audio performance.

There is further a pulsed electromagnetic field (PEMF) generator for delivering low-frequency PEMF waves to the user at known healing frequencies. According to the instant principles, the delivery to the user of the above stimuli as a single, coherent or synchronized and immersive multisensory experience induces in the user a sense of participation in the multisensory experience.

In the preferred embodiment, the audio beat frequency is between 6 Hertz (Hz) and 100 Hz. In the same or a related embodiment, the audio beat frequency is 40 Hz. In the same or a related embodiment, the instant pod chamber or simply pod also comprises an olfactory diffuser that delivers aromas to the user in concert with the audio beat frequency and the audio performance. In the same or a related embodiment, there is a roof screen on which visuals are delivered to the user in concert with the audio beat frequency and the audio performance.

In the same or a related embodiment, the stroboscopic light source comprises of light emitting diodes (LEDs). In the same or a related embodiment, the LEDs produce light that spans the visible spectrum of natural sunlight. In the same or a related embodiment, the vibro-tactile transducers are provided in the chair on which the user is reclined. In the same or a related embodiment, there is at least one vibro-tactile transducer positioned under the lower spine, at least one under the gluteus maximus and at least one under the calves of the user reclined in the chair inside the instant pod.

Most preferably, the pod chamber comprises a door that is advantageously closed while the instant multisensory experience is delivered to the user. Preferably, the door is controlled by a string door mechanism. Preferably, the string door mechanism is equipped with an automatic release enhancement. Preferably still, there is also an air conditioning unit with air ducts to ventilate the pod and to maximize the comfort of the user during the immersive multisensory session.

The systems and apparatus of the present technology include a pod chamber for delivering a multisensory experience to a user reclined inside said pod chamber, said pod chamber comprising: (a) a first set of audio speakers behind the head of said user for delivering two coherent sound waves that produce an audio beat frequency; (b) a second set of audio speakers on each side of the head of said user for delivering an audio performance in concert with said audio beat frequency; (c) a stroboscopic light source for delivering a strobing pattern designed to be perceived by said user with eyes closed, said strobing pattern being in concert with said audio beat frequency and with said audio performance in order to produce a rapid eye movement in said user; (d) a set of vibro-tactile transducers for delivering tactile vibrations to said user in concert with said audio beat frequency and with said audio performance; (e) a pulsed electromagnetic field unit generator for delivering electromagnetic waves to said user; whereby said predetermined audio beat frequency, said audio performance, said strobing pattern, said tactile vibrations and said electromagnetic waves induce in said user a sense of participation in said multisensory experience.

The methods of the present technology include a method for delivering a multisensory experience to a user reclined inside a pod chamber, said method comprising the steps of: (a) delivering by a first set of audio speakers behind the head of said user, two coherent sound waves that produce an audio beat frequency; (b) delivering by a second set of audio speakers on each side of the head of said user, an audio performance in concert with said audio beat frequency; (c) delivering a strobing pattern by a stroboscopic light source designed to be perceived by said user with eyes closed, said strobing pattern being in concert with said audio beat frequency and with said audio performance in order to produce a rapid eye movement in said user; (d) delivering tactile vibrations by a set of vibro-tactile transducers to said user in concert with said audio beat frequency and with said audio performance; (e) delivering electromagnetic waves by a pulsed electromagnetic field generator unit to said user; and (f) inducing in said user a sense of participation in said multisensory experience as a result of said audio beat frequency, said audio performance, said strobing pattern, said tactile vibrations and said electromagnetic waves.

Clearly, the system and methods of the invention find many advantageous embodiments. The details of the invention, including its preferred embodiments, are presented in the below detailed description with reference to the appended drawing figures.

### BRIEF DESCRIPTION OF THE DRAWING FIGURES

FIG. 1 illustrates an isometric view of a pod chamber for providing a multisensory experience to a user reclined in the pod according to the instant principles.

FIG. 2A-F show various views of the exterior of an instant pod chamber.

FIG. 3A illustrates a cutaway right side-view of an instant pod, showing its interior and the various components contained therein.

FIG. 3B shows the view of FIG. 3A in a fully rendered form.

FIG. 3C illustrates a cutaway left side-view of an instant pod, showing its interior and the various components contained therein.

FIG. 3D shows the view of FIG. 3C in a fully rendered form.

FIG. 3E illustrates a cutaway back view of an instant pod, showing its interior and the various components contained therein.

FIG. 3F shows the view of FIG. 3E in a fully rendered form.

FIG. 3G illustrates a cutaway front view of an instant pod, showing its interior and the various components contained therein.

FIG. 3H shows the view of FIG. 3G in a fully rendered form.

FIG. 3I illustrates a cutaway top view of an instant pod, showing its interior and various components in a fully rendered form.

FIG. 4A-E show screenshots from a graphical user interface (GUI) available to a user of an exemplary implementation of the present technology.

FIG. 4F-G show screen shots from a GUI available to an admin/technician of an exemplary implementation.

FIG. 5A shows the first or landing page/screen of the Session mode of an exemplary implementation of the present technology.

FIG. 5B shows the screen for Demo sub-mode of an exemplary implementation.

FIG. 6A-B show the initial and confirmation screens of Recharge sub-mode of an exemplary implementation.

FIG. 7A-B show the initial and confirmation screens of Reconnect sub-mode of an exemplary implementation.

FIG. 8A-B show the initial and confirmation screens of Rejuvenate sub-mode of an exemplary implementation of the present technology.

FIG. 9A-B show the initial and confirmation screens of Relax sub-mode of an exemplary implementation.

FIG. 10A-B show the initial and confirmation screens of Recover sub-mode of an exemplary implementation.

FIG. 11A-B show the initial and confirmation screens of Custom Session mode of an exemplary implementation.

FIG. 12A-J show informational popups explaining the Custom mode and various sensory stimuli or outputs produced by the pod of an exemplary implementation.

FIG. 12K shows the screen on which the user can select their own audio track or musical piece from an audio library.

FIG. 13 shows the screen of an exemplary implementation allowing the user to save a preset configuration.

FIG. 14A-B show exemplary settings and preferences screens for the user of an exemplary implementation of the present technology.

FIG. 15 shows a computer architecture diagram of an instant pod system.

FIG. 16 illustrates a network diagram showing a network of pods based on the instant principles.

FIG. 17A-B show the door of the pod in a half open and a fully open position respectively while exposing its interior.

FIG. 18A-T illustrate various views of the string door mechanism of the present technology.

FIG. 19A-D illustrate the automatic release enhancement to the string door mechanism of FIG. 18A-T based on the instant principles.

### DETAILED DESCRIPTION

The figures and the following description relate to preferred embodiments of the present invention by way of illustration only. It should be noted that from the following discussion, alternative embodiments of the structures and methods disclosed herein will be readily recognized as viable alternatives that may be employed without departing from the principles of the claimed invention.

Reference will now be made in detail to several embodiments of the present invention(s), examples of which are illustrated in the accompanying figures. The figures depict embodiments of the present invention for purposes of illustration only. One skilled in the art will readily recognize from the following description that alternative embodiments of the structures and methods illustrated herein may be employed without departing from the principles of the invention described herein.

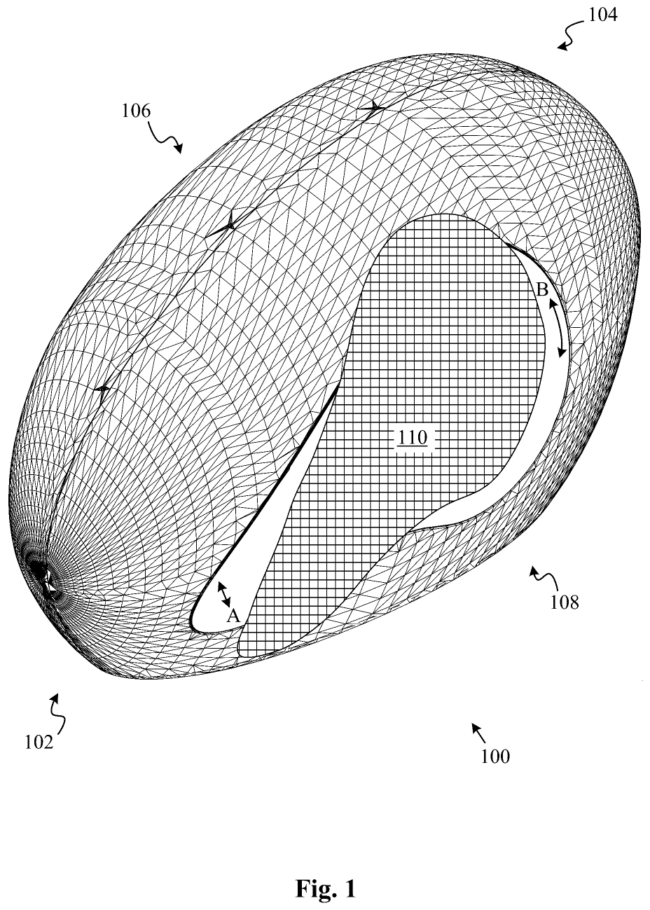

The systems and methods of the present invention will be understood by first reviewing a multisensory pod of a preferred embodiment as shown in FIG. 1. FIG. 1 is an isometric view illustrating a pod chamber 100 for delivering an immersive multisensory experience to a user according to the invention. Pod chamber 100 is generally oval shaped and has a front end 102, a back end 104, a top 106 and a bottom 108. To provide a completely enclosed environment for the user, pod chamber 100 is equipped with a door 110. In the preferred embodiment shown in FIG. 1, door 110 is on the left-hand side of pod 100. In alternative embodiments, door 110 may be on the right-hand side or on both sides. Referring to FIG. 1, door 110 is designed to recess into pod chamber 100 and open and close by rotating back and forth in directions A and B as shown. FIG. 1 shows pod chamber or simply pod 100 with its door 110 slightly ajar.

When pod chamber 100 is fully open, door 110 allows the user to enter. Then, by reversing the movements indicated by arrows A and B, door 110 closes off pod chamber 100 to provide a completely enclosed environment or enclosure to the user inside. In other words, door 110 is designed for closing off pod chamber 100 to achieve complete enclosure of the user reclined inside and thus place the user in a space that feels private and safe.

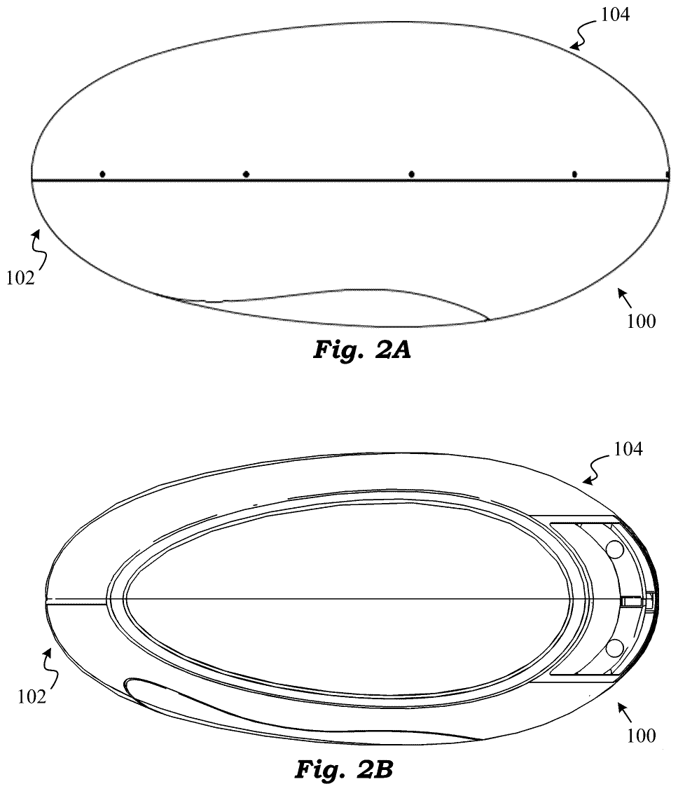

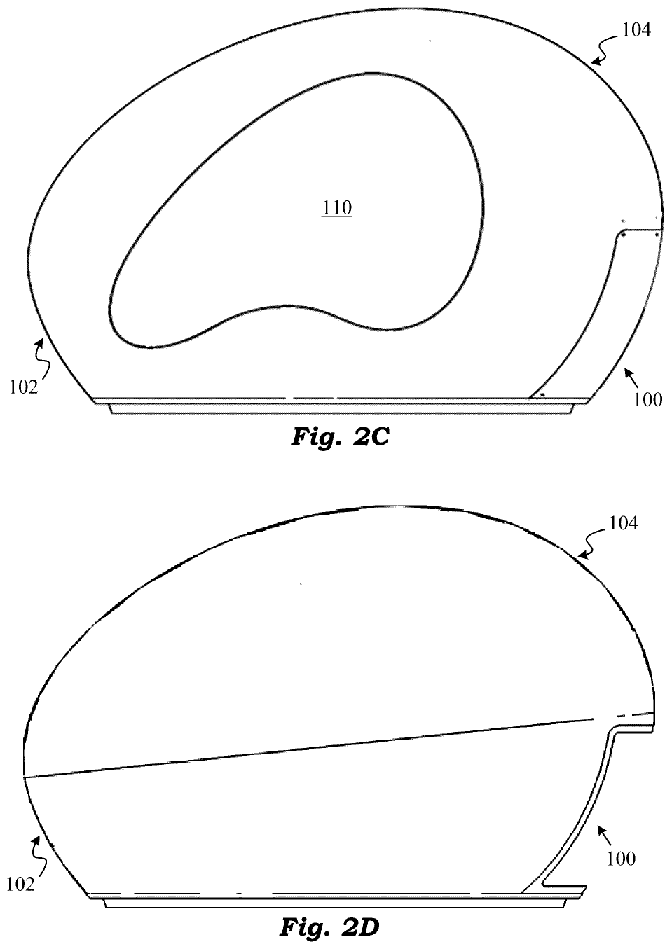

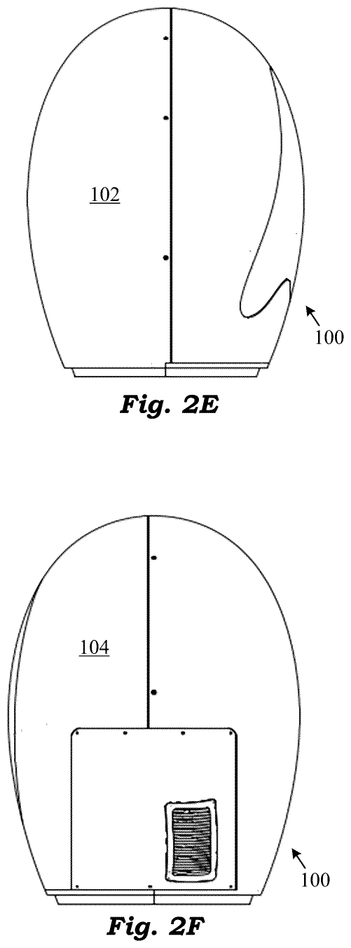

FIG. 2A and FIG. 2B show top view and bottom view respectively of the exterior of pod chamber 100. FIG. 2C and FIG. 2D show left side-view and right side-view respectively of the exterior of pod chamber 100. FIG. 2E and FIG. 2F show views of front 102 and back 104 respectively of the exterior of pod chamber 100 of the present invention.

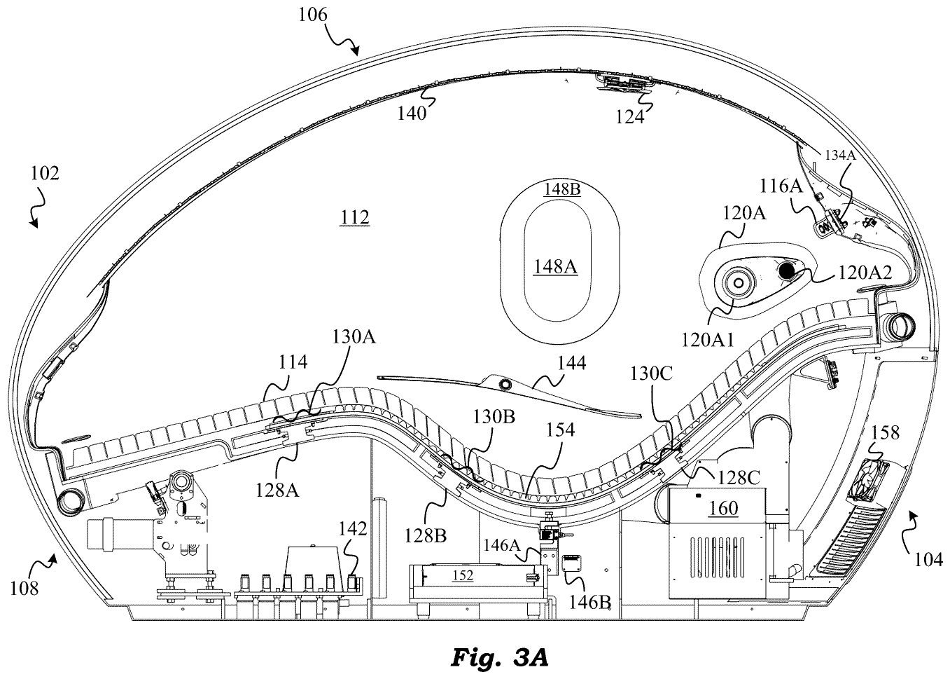

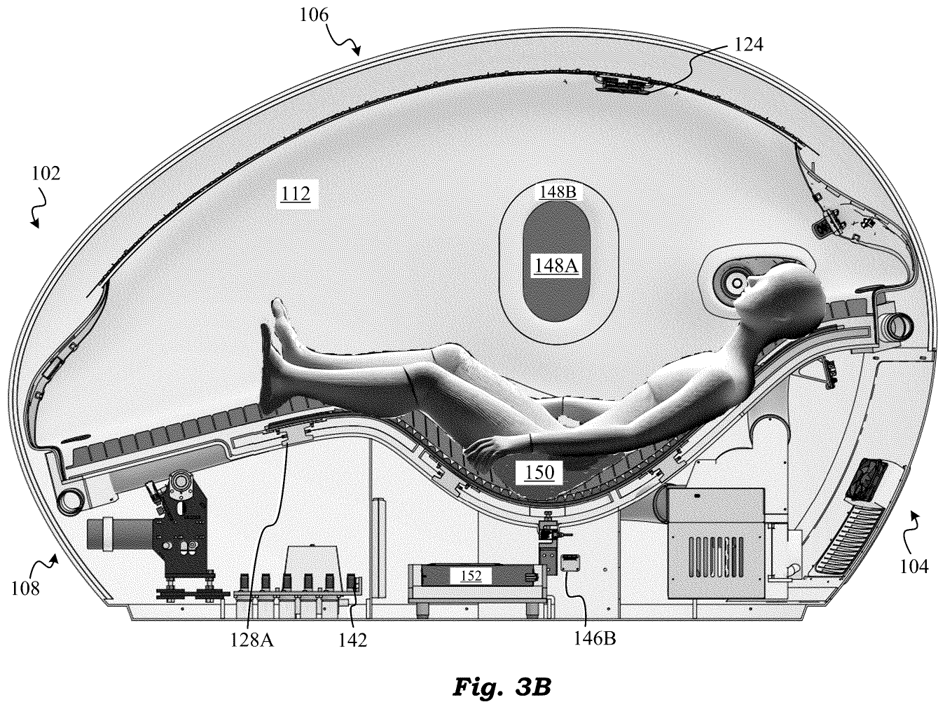

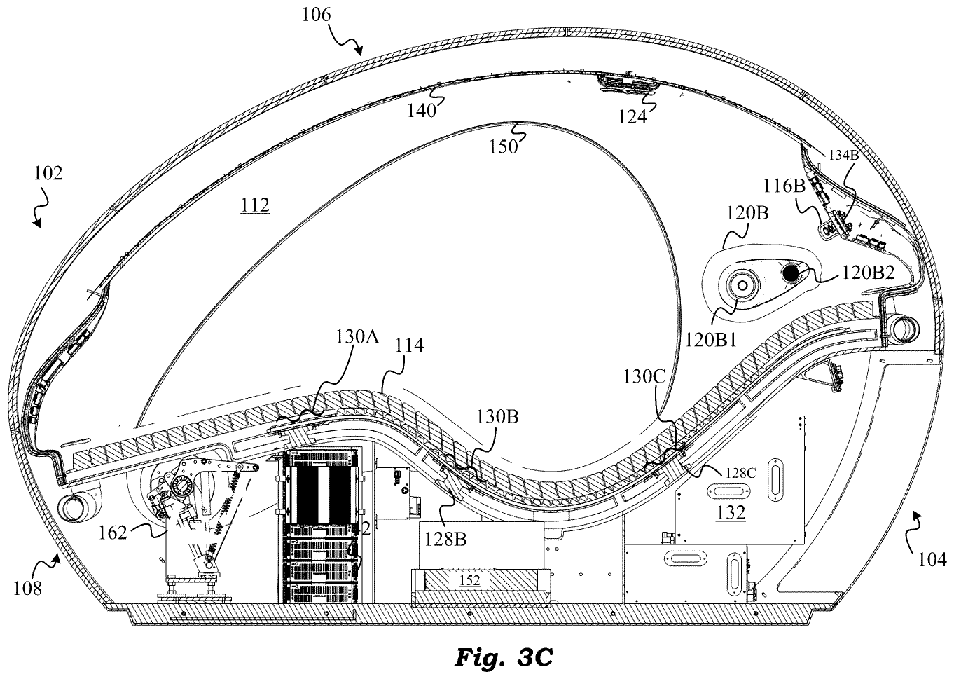

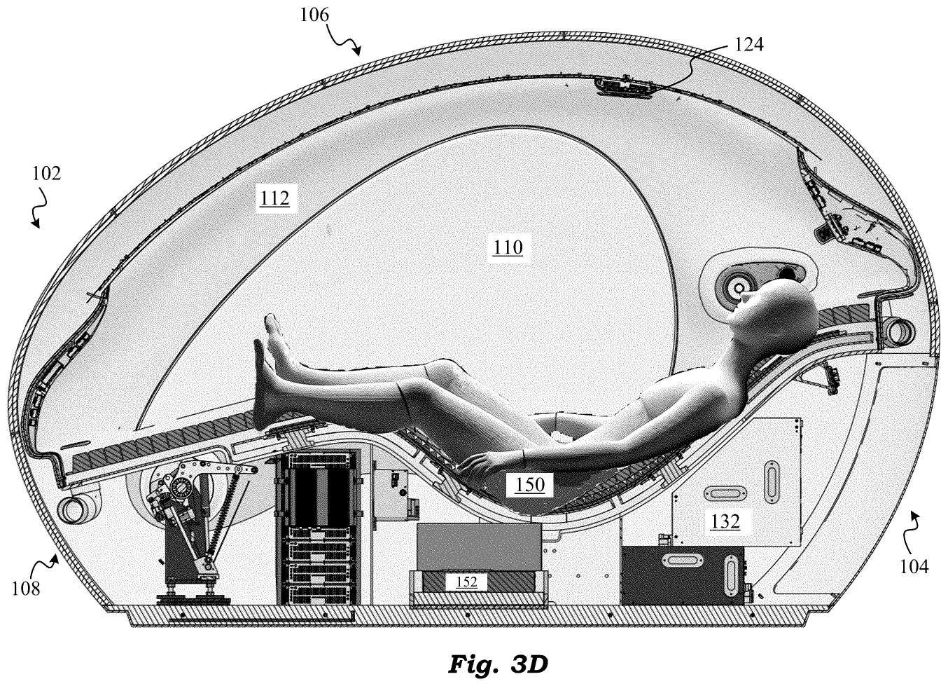

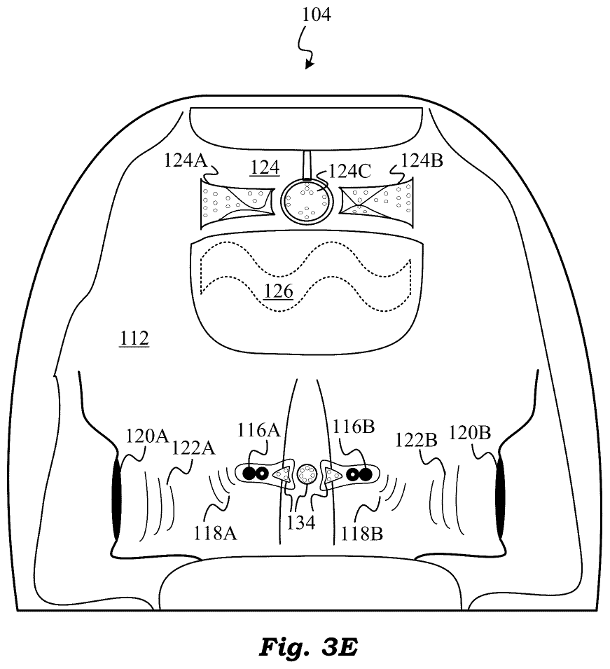

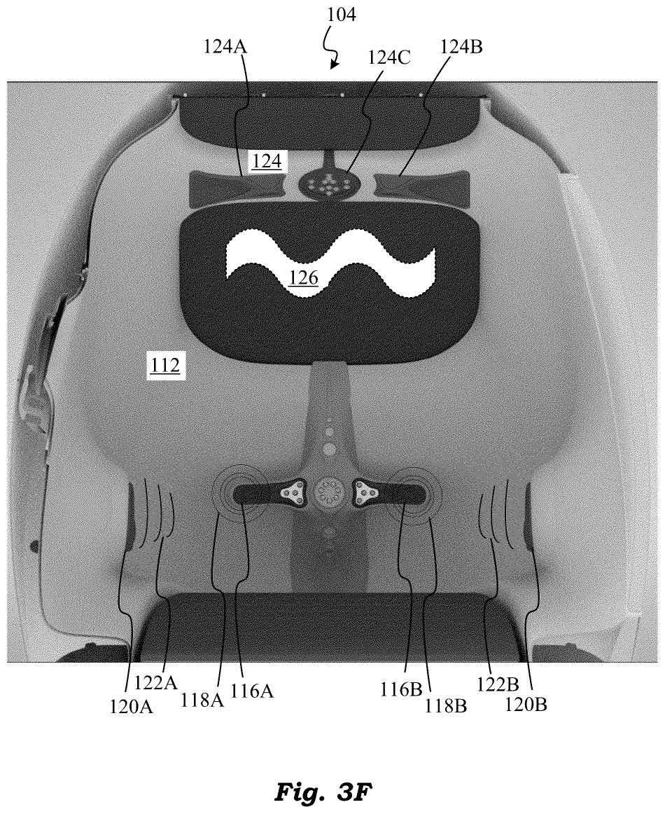

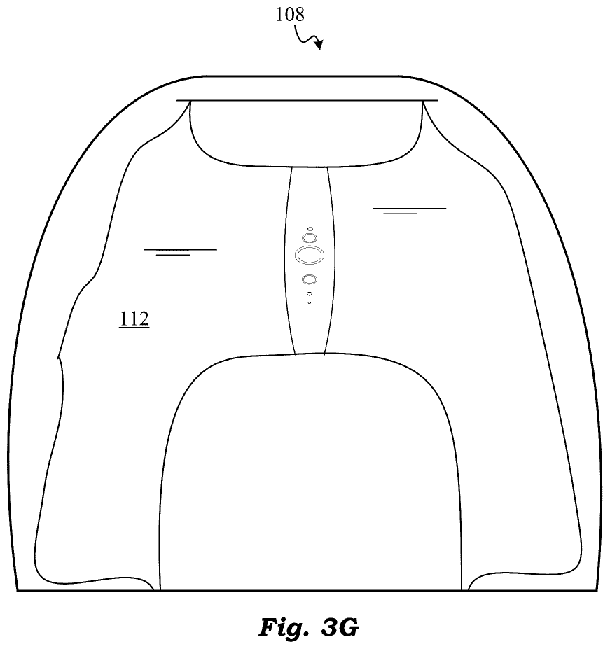

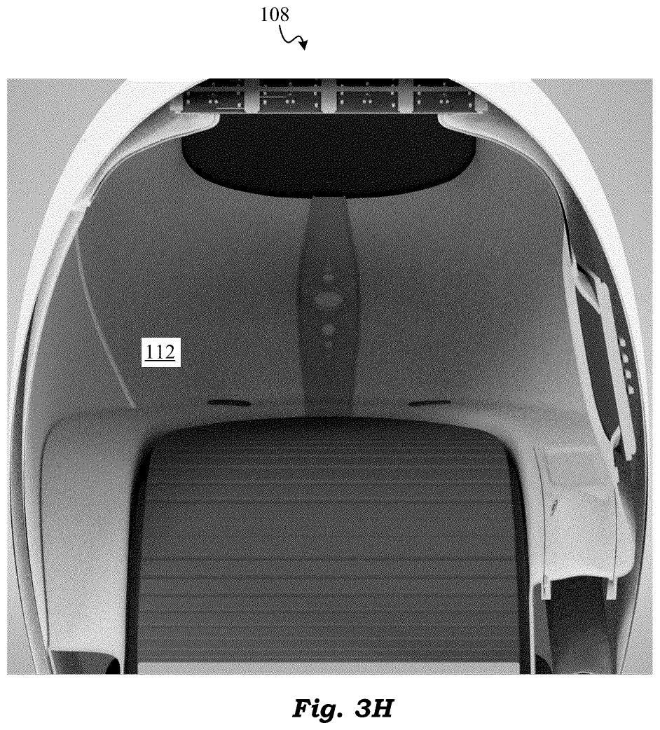

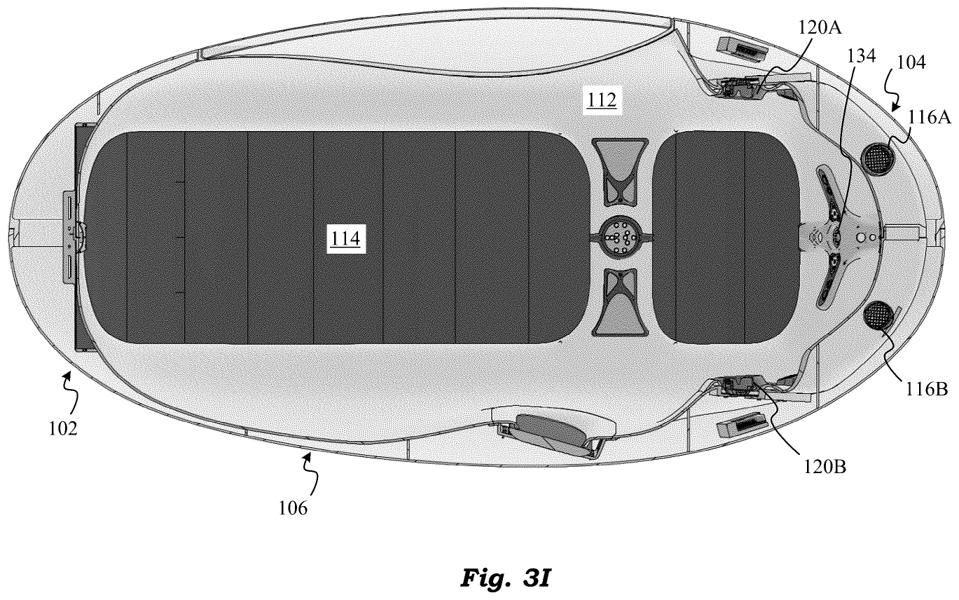

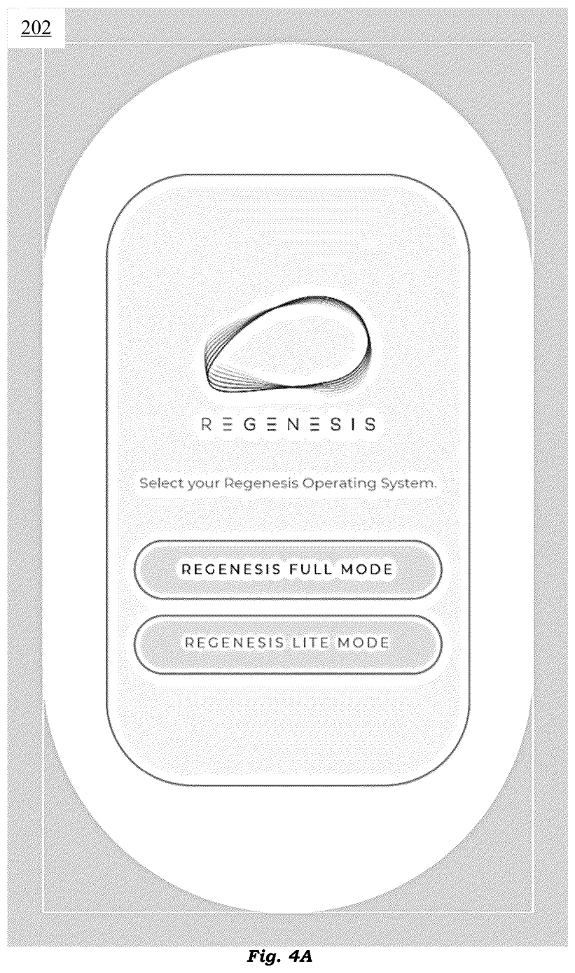

Now, let us take a look at the interior of pod 100 and its operation based on the present principles in greater detail. FIG. 3A shows a three-dimensional (3D) right side-view with a cutaway to expose interior 112 of pod chamber 100. For completeness, FIG. 3B illustrates a fully rendered version of FIG. 3A except that FIG. 3B also explicitly shows a user 150 reclined on chair or seat 114 as shown. In a similar manner, FIG. 3C shows a 3D left side-view with a cutaway to expose interior 112 of pod chamber 100. For completeness, FIG. 3D illustrates a fully rendered version of FIG. 3C except that FIG. 3D also explicitly shows a user 150 reclined on chair or seat 114 as shown. Just a few elements from FIG. 3A are marked in FIG. 3B and from FIG. 3C are marked in FIG. 3D in order to avoid clutter.

Note that door 110 is visible in the 3D left side-views of FIG. 3C-D but not in the right side-views of FIG. 3A-B. Furthermore, user 150 is only shown in the rendered views of FIG. 3B and FIG. 3D but not in the views of FIG. 3A and FIG. 3C in order to clearly show the various elements in interior 112 of the system that would have been otherwise obscured. FIG. 3C shows a door mechanism 162 of the present design that is responsible for the operation of door 110 for its opening and closing as discussed above.

A recliner chair or support or seat 114 is provided within interior 112 of pod 100 of FIG. 3 to accommodate user 150 in a reclined pose inside pod chamber 100 as shown in FIG. 3B and FIG. 3D. Interior 112 has a number of elements for delivering the immersive multisensory experience to user 150 reclined on recliner support 114 as will be explained further below.

The elements for delivering the present immersive multisensory experience or multisensory experience for short, comprise a first set of audio speakers that includes a left speaker 116A and a right speaker 116B. Preferably, each of speakers 116A and 116B is a set of speakers 116A and 116B respectively consisting of at least a woofer and a tweeter, however the woofers and tweeters of speakers 116 are not explicitly shown in FIG. 3 to avoid clutter. Only right audio speaker(s) 116A can be seen in the cutaway right side-view of FIG. 3A-B.

FIG. 3E and its corresponding rendered image of FIG. 3F show a view of back 104 of interior 112 depicting both right and left sets of audio speakers 116A, 116B respectively. FIG. 3G and its corresponding rendered image of FIG. 3H show a view of front 108 of interior 112 of pod 100. Again, not all the elements from FIG. 3E and FIG. 3G are explicitly marked in the rendered images of FIG. 3F and FIG. 3H respectively for reasons of clarity. For completeness, FIG. 3I shows a cutaway view of top 106 of pod 100 exposing its interior 112. Again, only some of the elements from earlier drawing figures including speakers 116 and 120 are marked in FIG. 3I to avoid clutter. Front 102, back 104 and recliner/chair 114 of pod 100 are also marked in FIG. 3I as shown.

The instant arrangement of the speakers ensures that audio speakers 116 are on each side behind the head of user 150 when in the proper reclined pose shown in FIG. 3B and FIG. 3D. This first set of audio speakers 116A, 116B is designed for delivering two respective coherent sound waves or tones that are schematically visualized and marked by reference numerals 118A, 118B in FIG. 3E-F. These sound waves combine to produce a certain audio beat frequency or binaural beat according to the instant design.

To produce the instant audio beat frequency, the two coherent sound waves/tones 118A, 118B need to be at slightly different frequencies. In other words, the two coherent sound waves 118A, 118B need to have a frequency offset. The superposition of such two coherent waves 118A, 118B with frequency offset (where the offset is equal to the difference between their frequencies), produces an audio beat frequency. The beat frequency is equal to the offset (also sometimes called the envelope of the superposition). This effect is referred to as binaural beats and the audio beat frequency is the frequency of the binaural beat.

Further explained, a binaural beat is an auditory illusion or a “phantom” beat perceived by the user when two different auditory pure-tone sine waves at a frequency offset are presented simultaneously. Advantageously, the audio beat frequency or the binaural beat frequency or simply the binaural beat is chosen to lie between 6 Hertz (Hz) and 100 Hz. At or above 100 Hz, a typical human user will no longer perceive any binaural beats. In the most preferred embodiment, the audio beat frequency is chosen to be at 10 Hz or at substantially 10 Hz.

In another embodiment, the audio beat frequency or the binaural beat is at 40 Hz or at substantially 40 Hz. In such an embodiment, the 40 Hz frequency offset of the binaural beat is preferably created by delivering 100 Hz sound waves in left speaker 116B and delivering 140 Hz in sound waves in right speaker 116A. In an alternative embodiment however, the frequencies of the sound waves in the left speaker 116B and right speaker 116A are respectively 216 Hz and 256 Hz for generating the 40 Hz binaural beat. There are number of such frequency combinations for the left and right speakers conceivable for generating the instant binaural beat of a desired frequency.

A second set of audio speakers 120A, 120B is provided in interior 112 on the right and left sides respectively of the head of user 150. Each of speakers 120A and 12B preferably consist of a woofers 120A1 and 120B1 and tweeters 120A2, and 120B2 as shown in FIG. 3A and FIG. 3C. Second set of audio speakers 120A, 120B is also designed for delivering a part of the multisensory experience, namely an audio performance. The audio performance is schematically visualized and marked in FIG. 3E-F by reference numerals 122A and 122B emanating from speakers 120A and 120B respectively.

Audio performance 122 is generally any audio composition such as a musical piece, a song, an audio track or some still other type of audio performance. In the preferred embodiment, audio performance 122 delivered by second set of audio speakers 120A, 120B is in concert with the audio beat frequency or binaural beat perceived by user 150 per above explanation. The binaural beat of the audio beat frequency is produced as a result of offset coherent sounds waves 118A and 118B produced by speakers 116A and 116B respectively as discussed above.

To be considered in concert with the audio beat frequency, audio performance 122 needs to exhibit certain characteristic or characteristics. Specifically, audio performance 122 needs to incorporate the audio beat frequency as an integral part. This is accomplished by either including in audio performance 122 a base line that includes the audio beat frequency directly (as a direct part of the base line) or indirectly (not expressly included in the base line of audio performance 122 but relied upon to complete the base line).

Another way of ensuring that audio performance 122 is in concert with the audio beat frequency or the binaural beat is to provide a base line that includes higher harmonics of the audio beat frequency as an integral part. Depending on the embodiment and in still more advanced ways of thusly incorporating the binaural beat, a rhythm syncopation that either directly or indirectly uses the binaural beat can be integrated in audio performance 122. Skilled musical composers will be familiar with the nature of musical compositions and may find still other direct and indirect ways of incorporating, working with, or working off from the audio beat frequency. These approaches are all considered to present a concert between audio performance 122 and the audio beat frequency according to the instant principles.

A stroboscopic light source 124 is also preferably provided in interior 112 above the head of user 150 of FIG. 3. Stroboscopic light source 124 is also designed for delivering a part of the multisensory experience, namely a strobing pattern of light 126 visualized schematically in FIG. 3E-F. In the preferred embodiment, strobing pattern of light 126 is also in concert with the binaural beat frequency as well as the audio performance. In the same or a related embodiment, stroboscopic light source 124 is configured from a set of light emitting diodes (LEDs). The light emitted from the LEDs of stroboscopic light source 124 preferably spans the full spectrum of visible wavelengths and replicates light as close to natural sunlight as possible.

In a preferred embodiment, the LEDs of light source 124 are arranged in a set of three panels, a right panel 124A, a left panel 124B and a center/central panel 124C as shown in FIG. 3E-F. In the same or another embodiment, additional LEDs are provided on a top panel or bar beside the stroboscopic light source 124. These LEDs advantageously produce light in the ultraviolet (UV) ranges UV-A, UV-B and UV-C and are thus UV LEDs. These are not explicitly marked in FIG. 3E-F for clarity but are present alongside the stroboscopic LEDs of stroboscopic light source or lamp 124.

UV-C are not used while the user is in pod chamber 100 as these high-energy electromagnetic waves are harmful to humans. The UV-C LEDs are instead used for sanitizing pod chamber 100 when it is empty. In addition to UV LEDs, one or more LEDs 134 in the near-infrared (near-IR) range are also provided in a preferred embodiment. These near-IR LEDs 134 are located behind the head of the user and preferably as a right set of one or more LEDs 134A and as a left set of one or more LEDs 134B as shown in FIG. 3A and FIG. 3C respectively.

It is important for stroboscopic light source 124 to be sufficiently bright to enable the user to perceive it with eyes closed. In fact, in the preferred mode the user does not open eyes to look directly at stroboscopic light source 124 during audio performance 122. The strobing pattern of light produced by strobing light source 124 alone or in combination with UV and/or near-IR LEDs is schematically visualized and marked by reference numeral 126 in FIG. 3E-F.

In accordance with the invention, strobing pattern of light 126 is also in concert with the audio beat frequency as well as with audio performance 122. In other words, strobing pattern of light 126 incorporates the audio beat frequency or the instant binaural beat as an integral part in the manner described above (e.g., by being integrated directly or indirectly within the rhythm and/or rhythm syncopation). Furthermore, strobing pattern of light 126 is also in concert with audio performance 122 by being integrated with it as well. This means that audio performance 122 and strobing pattern of light 126 are mutually integrated and form a joint composition using rhythms and/or themes. Such joint composition induces a desired effect on the user, namely rapid eye movement (REM).

As shown in the preferred embodiment of FIG. 3A, a set of vibro-tactile transducers 128A, 128B and 128C is also provided for user 150 shown in FIG. 3B inside pod chamber 100. In order to accommodate this placement, vibro-tactile transducers 128 are mounted below recliner support or chair 114, as illustrated in FIG. 3A and FIG. 3C. Vibro-tactile transducers 128A, 128B, 128C are designed for delivering tactile vibrations are that schematically visualized and marked by reference numerals 130A, 130B, 130C respectively to the user while in the reclined pose on recliner support 114.

In the preferred embodiment shown in FIG. 3, set of vibro-tactile transducers 128A, 128B, 128C include one or more transducers 128C positioned under the lower spine, one or more transducers 128B positioned under the gluteus maximus, and one or more transducers 128A positioned under the calves. Of course, other placements can also be devised in other embodiments of the invention and based on the instant principles.

It is important to ensure that tactile vibrations 130A, 130B, 130C are also an integral part of the multisensory experience. In other words, tactile vibrations 130A, 130B, 130C are also preferably delivered in concert with the audio beat frequency and with audio performance 122. This is accomplished in the manner described above by rhythmic and/or thematic incorporation with audio performance 122 and incorporation with the audio beat frequency (e.g., direct or indirect rhythmic integration and/or rhythmic syncopation).

A pulsed electromagnetic field (PEMF) generator unit 132 is also provided in interior 112 underneath the user reclined inside pod chamber 100, as illustrated in FIG. 3C-D. PEMF generator unit 132 includes all the necessary elements (e.g.,, antennas, amplifiers, filters, coils, other electrical and electronic circuitry, etc.) for delivering pulsed low-frequency electromagnetic waves to user 150.

Preferably, these pulsed electromagnetic waves are delivered in the range of 1 Hz and 100 Hz. Preferably still, these pulsed electromagnetic waves are delivered at 40 Hz or at substantially 40 Hz. Preferably still, these pulsed electromagnetic waves are delivered at 10 Hz or at substantially 10 Hz. The pulsed low-frequency electromagnetic waves are delivered from PEMF generator unit 132 to the user at known healing frequencies. Although low-frequency electromagnetic waves are not directly perceivable to the user, it is known that the user will still receive the benefits of exposure to these pulsed electromagnetic fields. Preferably still, the PEMF waves may be delivered to user 150 in concert with the binaural beat and/or audio performance 122.

In accordance with the invention, the desired effect on the user is accomplished when the combination of the audio beat frequency, audio performance 122, strobing pattern 126, tactile vibrations 130 and electromagnetic waves delivered by PEMF generator unit 132 are all in concert or reconciled with each other as a singular, joint or synchronized and immersive multisensory The delivery of such concerted and immersive experience. multisensory experience is successfully accomplished when it induces in the user a sense of participation in the multisensory experience. We refer to the audio beat frequency, audio performance 122, strobing pattern 126, tactile vibrations 130, electromagnetic waves delivered by PEMF 132, or any other type of stimulation produced by pod 100 for user 150 as part of the above multisensory experience, as respective stimuli produced/generated/output by pod 100.

In various embodiments, pod chamber 100 of the invention includes additional elements to augment the multisensory experience. For example, in some embodiments pod chamber 100 has an olfactory aromatic diffuser for delivering aromas into interior 112. Such an olfactory diffuser 142 is shown in FIG. 3A-B. Again, the delivery of the aromas is carried out in concert with the beat frequency as well as with audio performance 122 in a similar manner as the delivery of other multisensory outputs or stimuli discussed above.

Olfactory diffuser 142 is preferably a standard essential oil diffuser which is triggered at specific points of time during the multisensory experience or session to promote and link the highest states of bliss and joy to a particular scent. It is widely understood that our olfactory system is able to trigger the strongest memories. The present design links the best moments during the multisensory experience in pod chamber 100 to the preferred scent of user 150.

An objective of pod 100 of the above teachings is to stimulate all of the senses, delivering a unique experience to user 150. As the pod brings the user to a heightened sense of euphoria and bliss, a particular scent is released from olfactory diffuser 142 into pod 100 that couples the experience with that particular smell. Then later, in times of stress and anxiety, the user can smell the same scent and be ‘taken back’ to that enjoyable memory of bliss and forget about their current stresses.

In the same or related embodiments, pod chamber 100 also has a roof screen 140, as seen in FIG. 3A and FIG. 3C, for delivering visuals to user 150. Once again, these visuals are preferably delivered in concert with the beat frequency or binaural beat as well as with audio performance 122. For example, some visuals are audio-reactive visuals, i.e., cymatics which emulate how audible sounds can be visually interpreted.

In the same or related embodiments, there is also a load sensing mechanism consisting of a load sensor or loadcell 146A with its associated driver 146B shown in FIG. 3A. Load sensing mechanism 146 automatically detects that user 150 has seated on chair 114. In the same or related embodiments pod chamber 100 also contains air conditioning capabilities to maximize user comfort. In the preferred embodiment shown in FIG. 3A, there is an air conditioning unit 160 and associated air ducts for circulating air and ventilating interior 112.

The various functions of the systems are controllable by user 150 via a human machine interface (HMI) 148 provided in interior 112 of pod 100. More specifically, HMI 148 consists of a touch-sensitive display or touchscreen 148A with its bezel 148B as shown in right side-views of FIG. 3A-B. Touchscreen 148A allows user 150 to touch/activate various graphical user interface (GUI) buttons or widgets in order to control the behavior of pod 100. Relatedly, there is also a manual console or simply a console 144 that consists of physical keys or knobs for issuing commands to the system. HMI 148 alone or in combination with console 114 allows the user to configure the multisensory experience obtained from pod of 100 of the present design as discussed further below.

In one embodiment, the configuration of the various mechanisms of pod 100 for delivering the multisensory experience to user 150 in order to produce a sense of participation in the multisensory experience, is preset or fixed. Preferably, the preset/fixed configuration or simply the preset would dictate the specific frequency of the audio beat frequency or binaural beat output via speakers 116. Preferably, such a preset would also determine whether or not an audio soundtrack is to be played via speakers 120 and if so which specific music or audio soundtrack.

Preferably, such a preset would also determine whether or not stroboscopic lights 124 are to be activated and if so which specific stroboscopic pattern 126 is to be output and at which specific frequency. Preferably, such a preset would also determine whether or not UV LEDs as well as near-IR LEDS 134 discussed above are to be activated and if so at what frequencies and for outputting which specific patterns. Preferably, such a preset would also determine whether or not vibrotactile transducers 128 are to be activated and if so in what order or sequence or arrangement. Preferably, such a preset would also determine any other configuration parameters governing the multisensory experience of the user while on seat 114 and using pod chamber 100 of FIG. 1-3.

However, in the preferred embodiment, the various parameters for controlling the user experience are configurable by the user before and during their multisensory experience in pod 100. For this purpose, the user can manipulate the widgets and navigate the various screens of the instant GUI displayed on HMI 148 and the various knobs/keys of console 144. This interactive capability allows the user to control the functions of pod 100, including starting and stopping the experience, configuring the various sensory stimuli and even wirelessly attaching their own personal computing device. In such a scenario, the user can play back audio and/or video content from their personal device e.g. a smartphone, to speakers 120 and roof screen 140 respectively if pod 100.

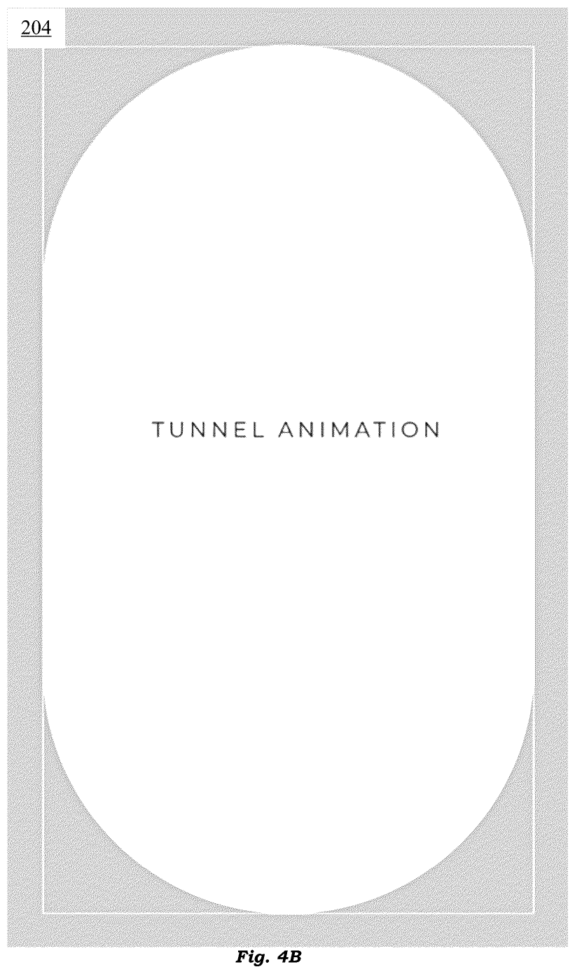

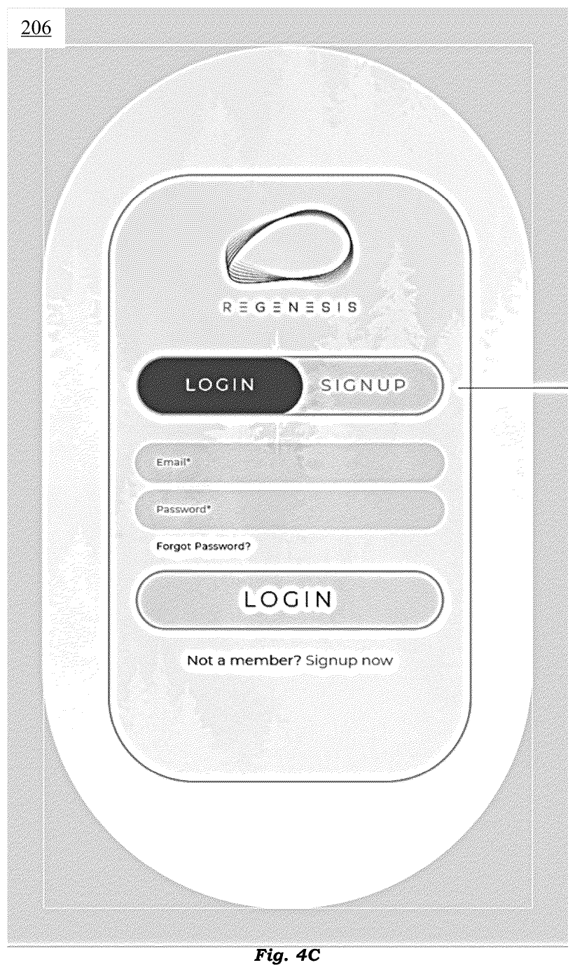

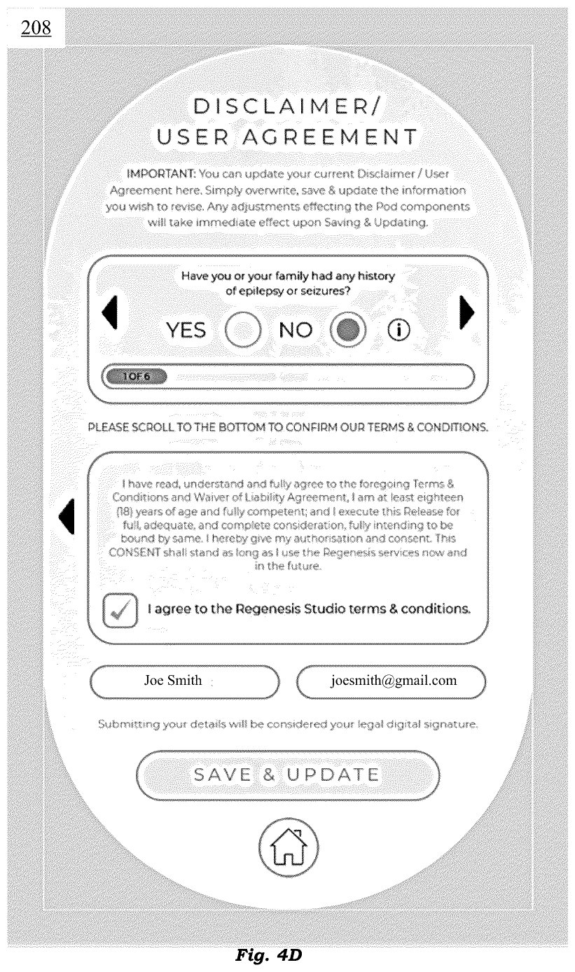

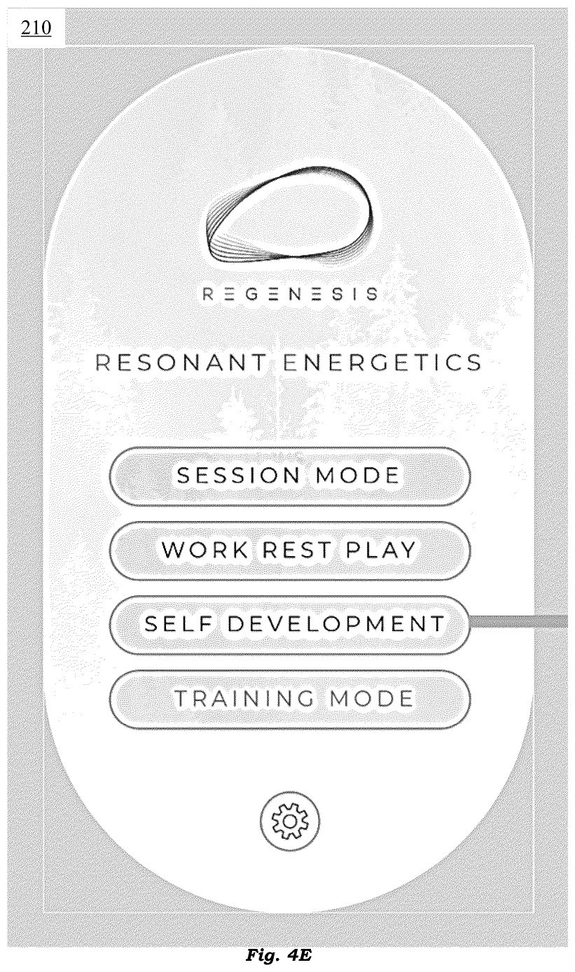

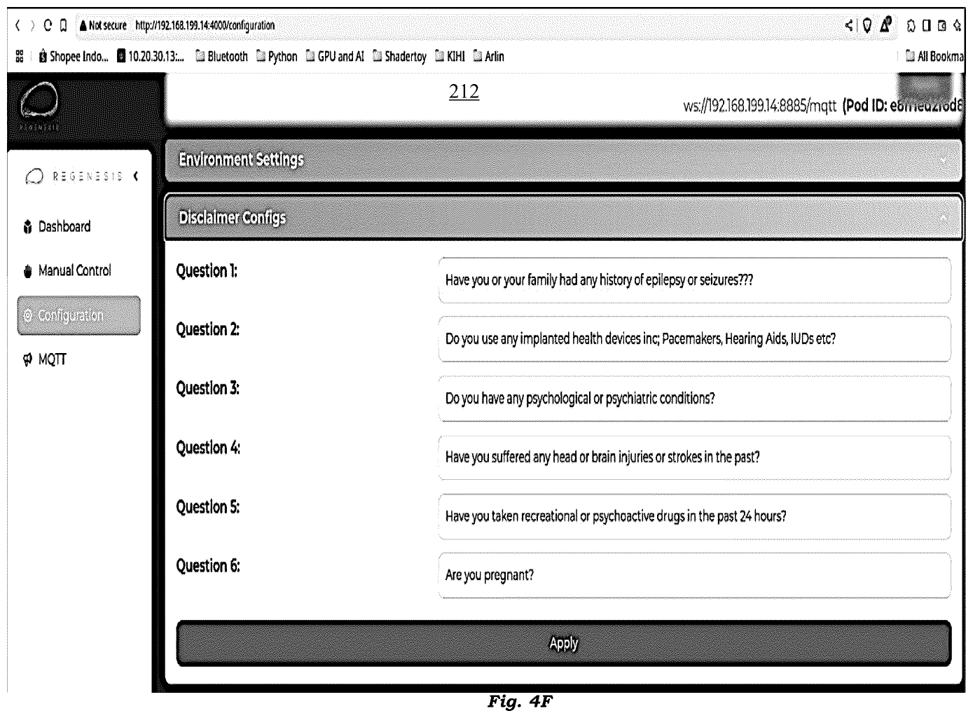

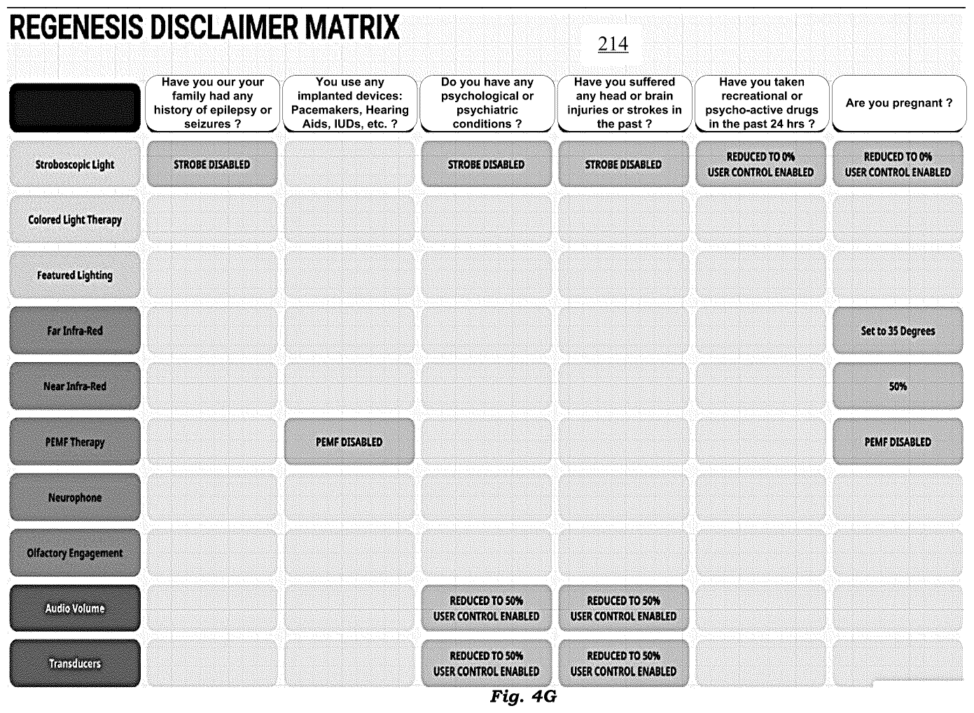

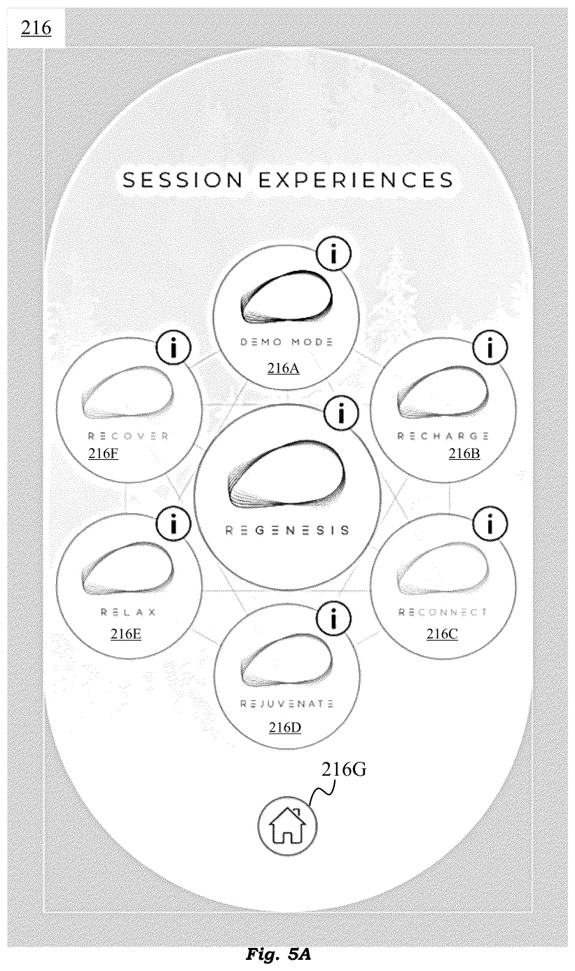

FIG. 4-14 show various screenshots or simply screens provided in the above-discussed GUI in a preferred implementation. The exemplary screenshots of the implementation are shown in FIG. 4-14 in grayscale on a white background with a dark foreground in order to improve legibility. However, in alternative embodiments, the foreground may be light or white in color and the background may be dark or black.

Let us now walkthrough the key user interface (UI) or user experience (UX) flows of the present design available via HMI 148 and console 144 to user 150 in great detail. FIG. 4A illustrates the first or welcome screen 202 that is shown by default on touch-sensitive HMI display 148A as user 150 first enters pod 100 of FIG. 3. FIG. 4B shows screen 204 displayed on HMI 148 when user 150 has seated on chair 114 as automatically detected by load sensing mechanism 146 discussed above. In a preferred embodiment, screen 202 of FIG. 4A is not shown and it is just screen 204 of FIG. 4B that is shown as the user sits down in the pod. Then, the user is shown screen 206 of FIG. 4C to sign-up for or login into the system as applicable. Once user 150 is logged in, the user is taken to screen 208 of FIG. 4D. Screen 208 and its associated popup screens or popups (not explicitly shown) are meant for the user to answer various questions about their health condition and history and electronically sign associated disclaimer form(s).

Once the user has signed the necessary disclaimer(s), then the user is presented with home screen 210 of FIG. 4E. Screen 210 displays the various modes of operation of pod 100, namely Session Mode, Work Rest Play mode, Self Development mode and Training mode. Most frequently, the Session mode is considered as the core experience for achieving the benefits of the multisensory experience by the users. As such, we will focus greatly on the various features and screens of the session mode in order to elucidate the innovative aspects of the present technology.

However, depending on the embodiment, the other modes cover traditional experiences, guided meditation, guided breathwork, sound healing, color therapy, among others. For instance, training mode includes biofeedback and artificial intelligence, such that the user can practice certain techniques in real-time based on the data that biofeedback wearables are able to capture. The guided meditation session mode specifically guides the user through a meditative state of mind.

For completeness, FIG. 4F shows screenshot 212 with an exemplary set of such disclaimer questions while FIG. 4F shows on screen 214 an exemplary configuration of various sensory stimuli of pod 100 that are enabled or disabled based on the answers to the disclaimer questions of FIG. 4E. All the stimuli are activated 100% by default unless otherwise noted as per the exemplary configuration shown in the disclaimer matrix of FIG. 4F. Screens 212 and 214 of FIG. 4F and FIG. 4G are preferably displayed to a technician or an administrator on an admin console and not to user 150 on HMI 148.

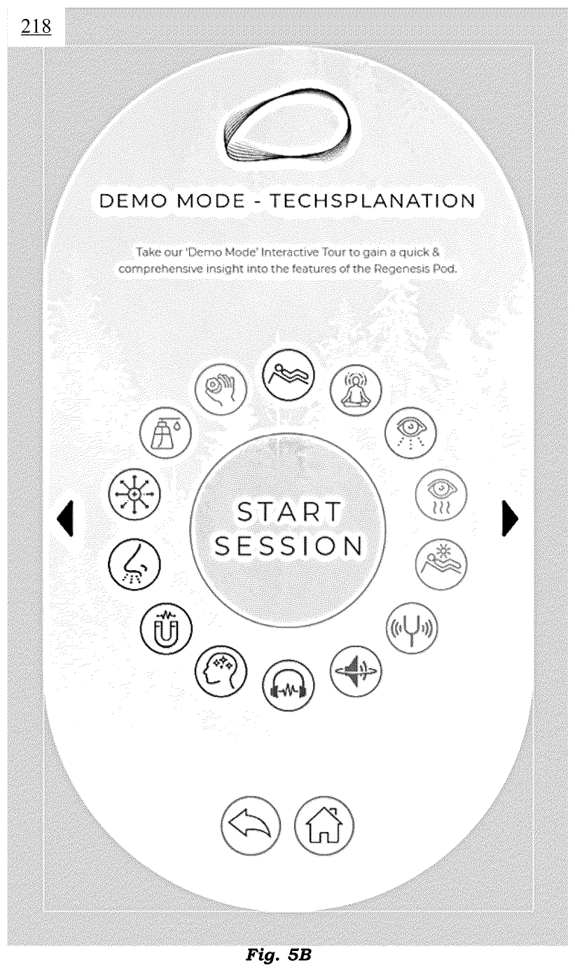

Once the user selects session mode on home screen 210, then the user is taken to the various screens of the session mode. Screenshot 216 of FIG. 5A shows the first or landing screen that is displayed to the user in the session mode. From screen 216, the user has the option of selecting the various sub-modes within the session mode of operation as shown. More specifically, and moving in a clockwise direction on screen 216, the user can select one of Demo Mode 216A, Recharge 216B, Reconnect 216C, Rejuvenate 216D, Relax 216E and Recover 216F sub-modes of operation of pod 100. At any point, the user can press or touch home button 216G to navigate back to home screen 210 discussed above.

If the user selects demo mode 216A from screen 216, screen 218 of FIG. 5B is displayed on touch-sensitive display 148A. Once the user presses/touches Start Session on screen 218, the demo mode is commenced for the user. Preferably, the objective of the demo mode is to demonstrate the various capabilities of the pod to user 150 in a comprehensive manner. Essentially, the demo mode is a specific preset configuration of the system that activates the various multisensory outputs/stimuli of pod 100 in a specific manner so as to provide a broad overview of the full capabilities of the system to user 150.

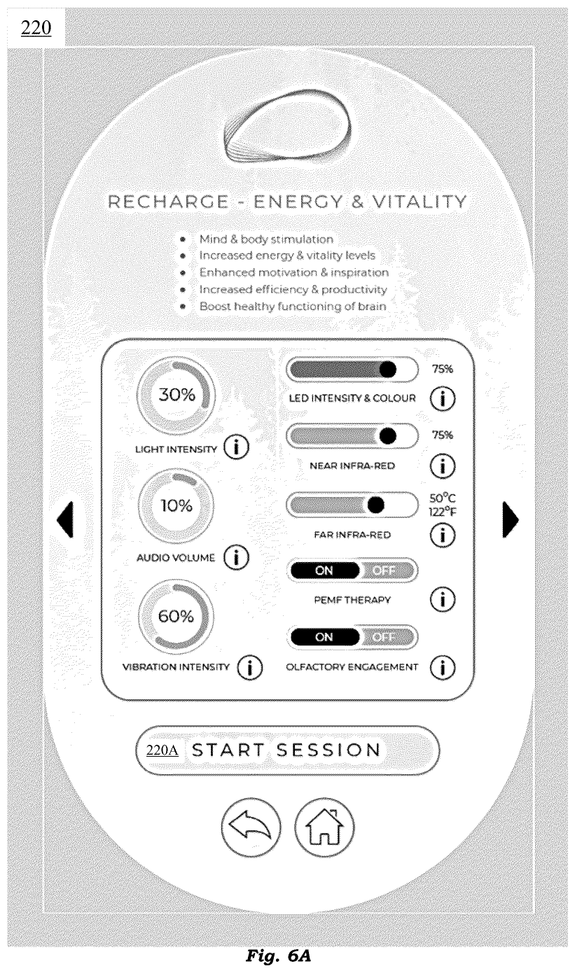

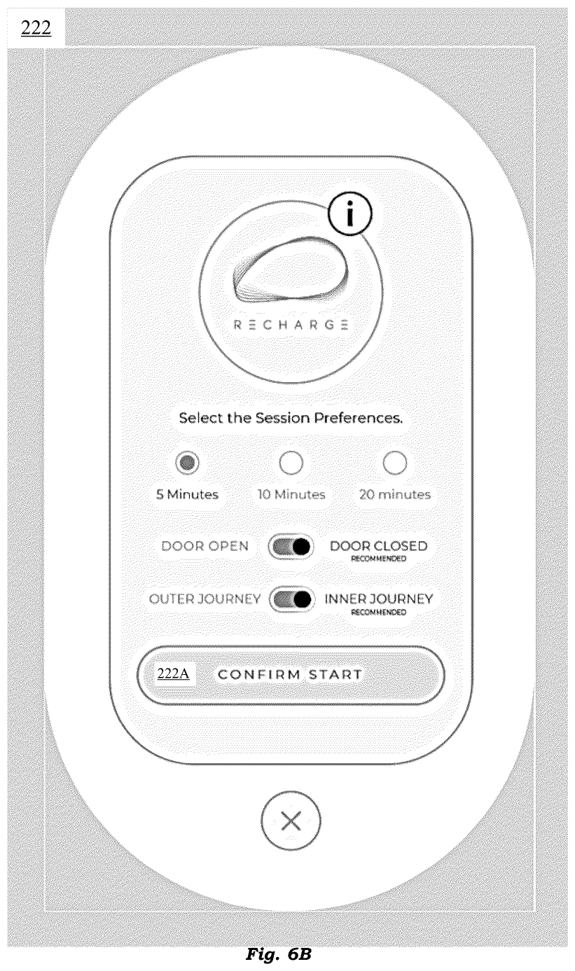

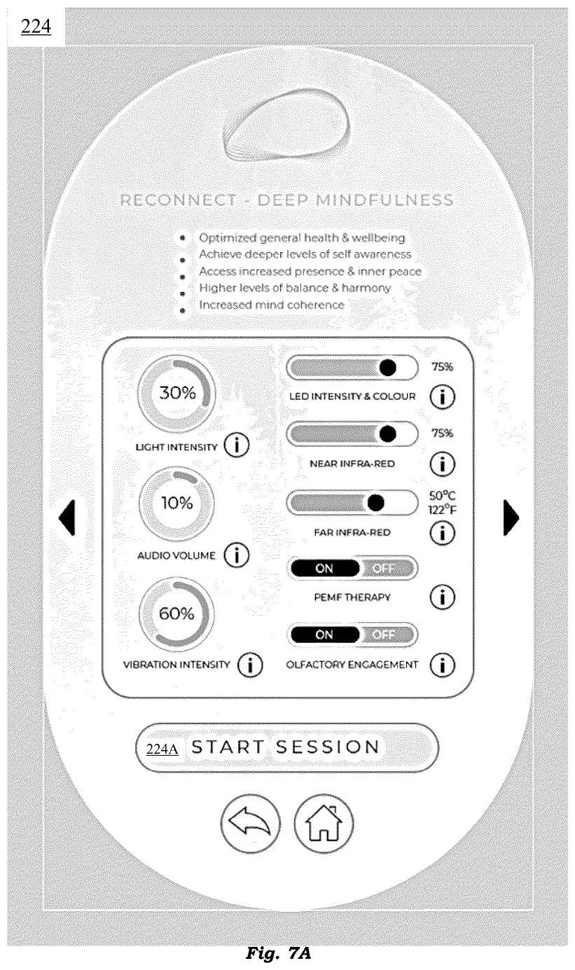

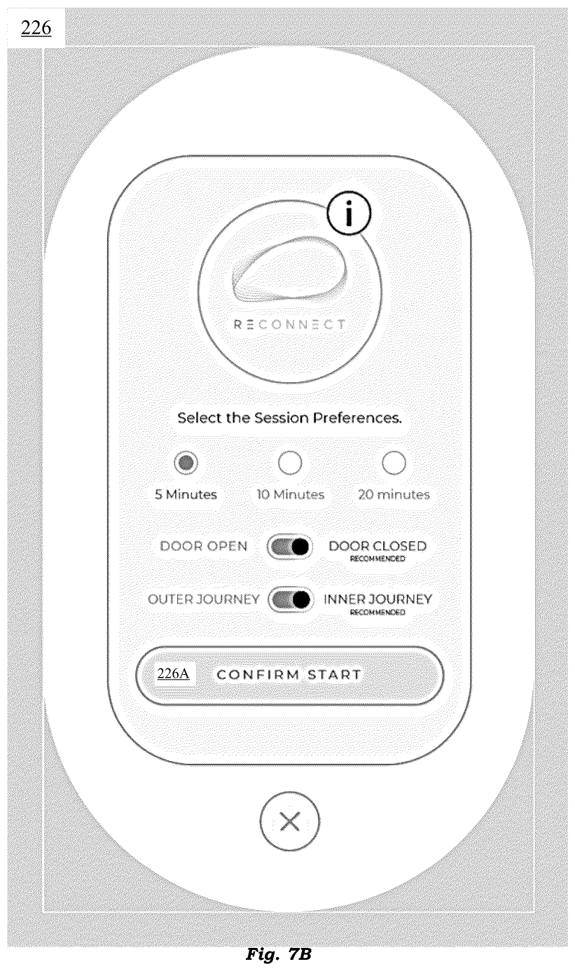

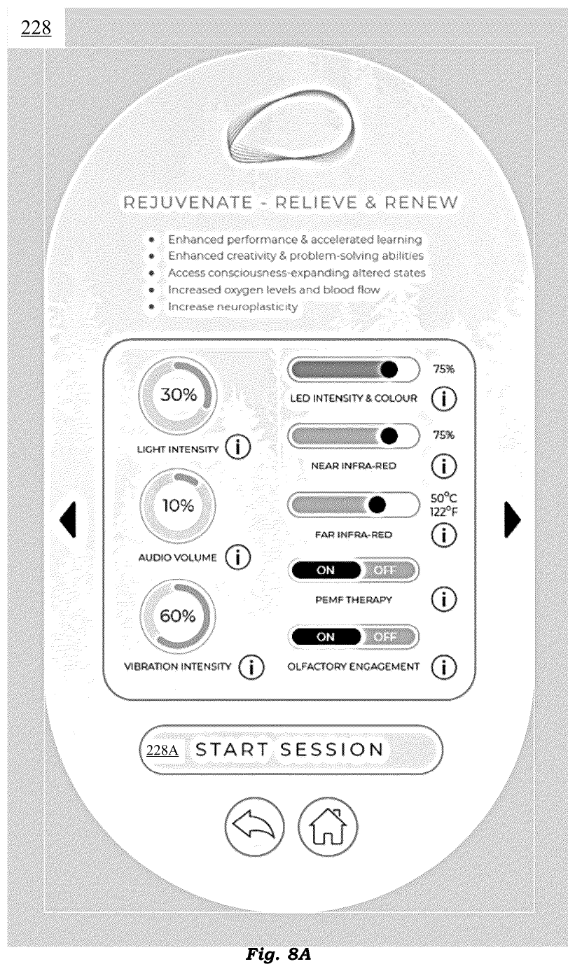

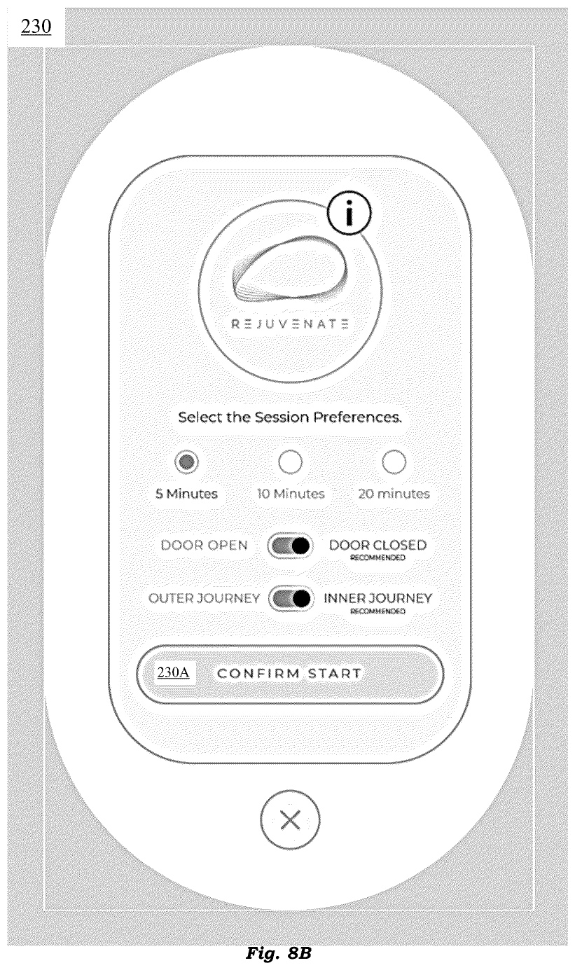

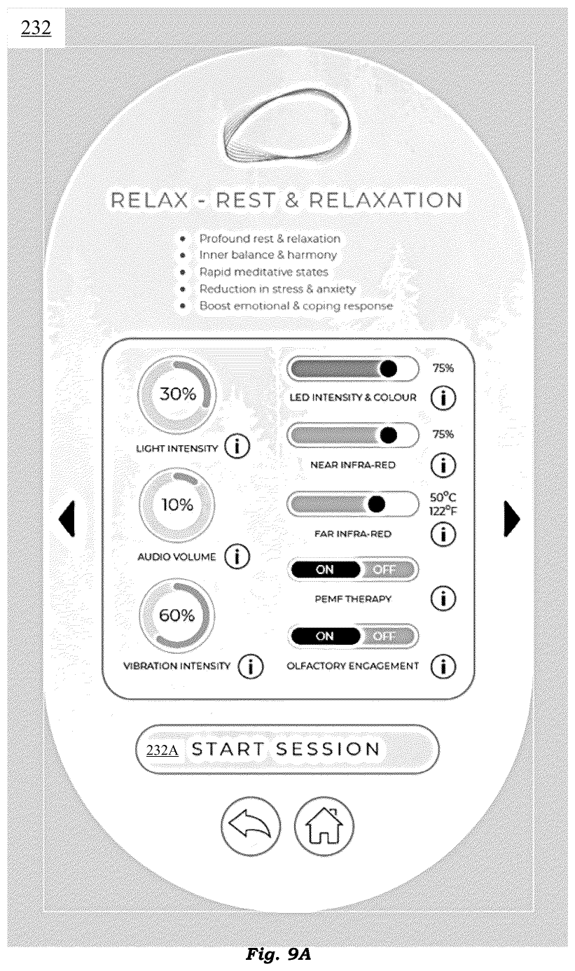

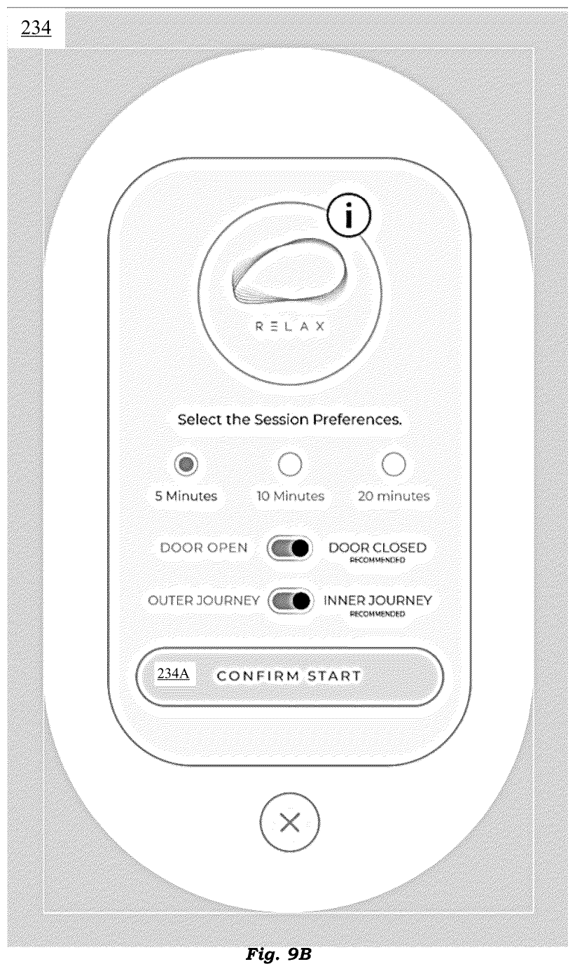

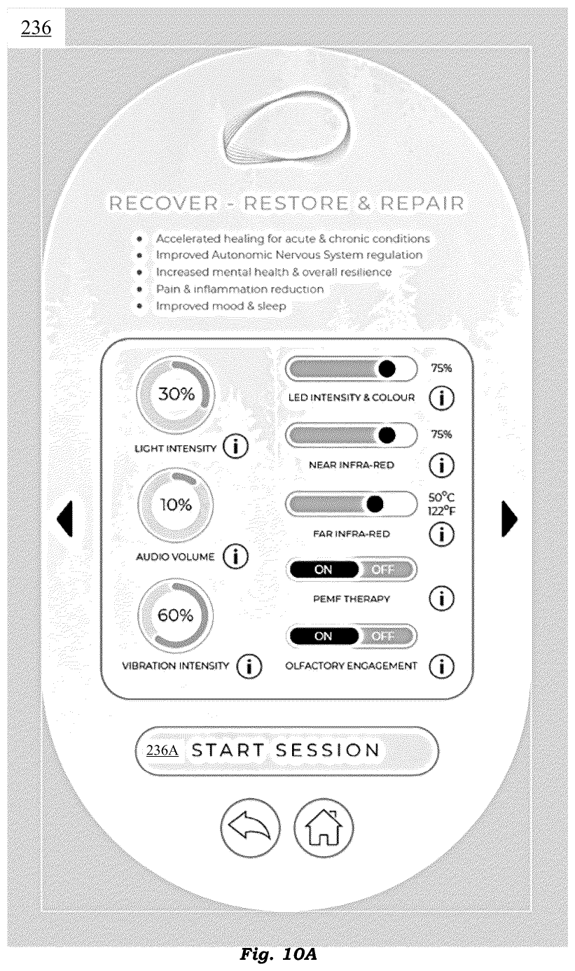

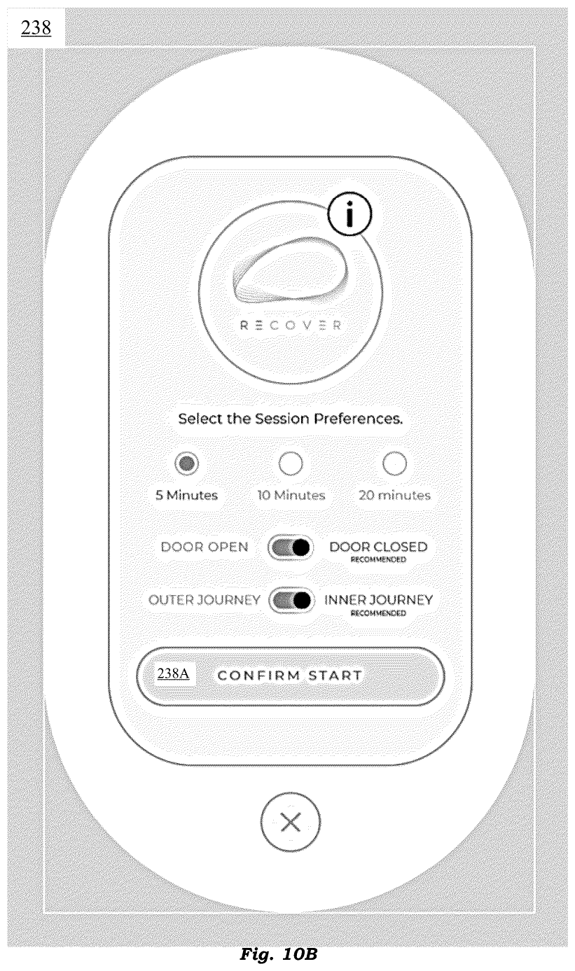

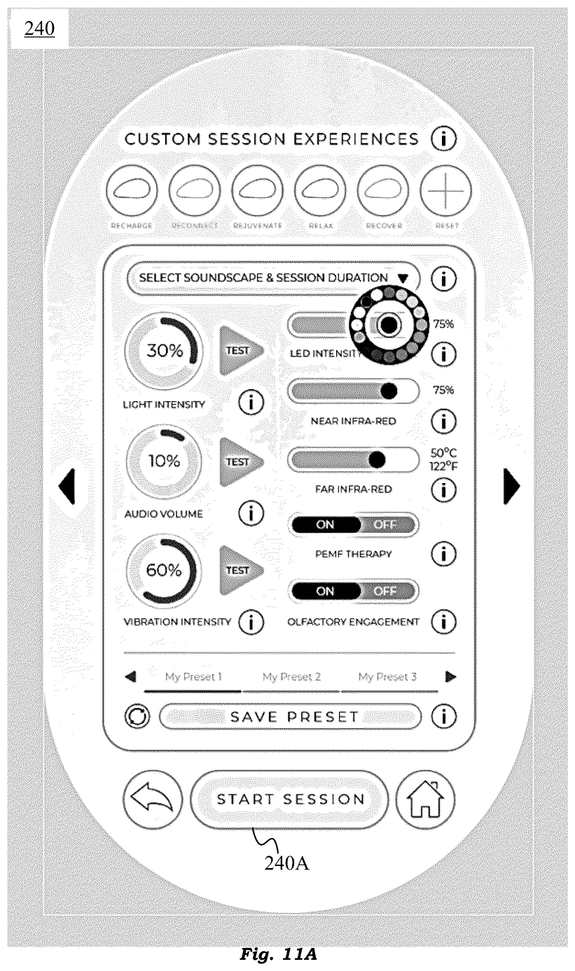

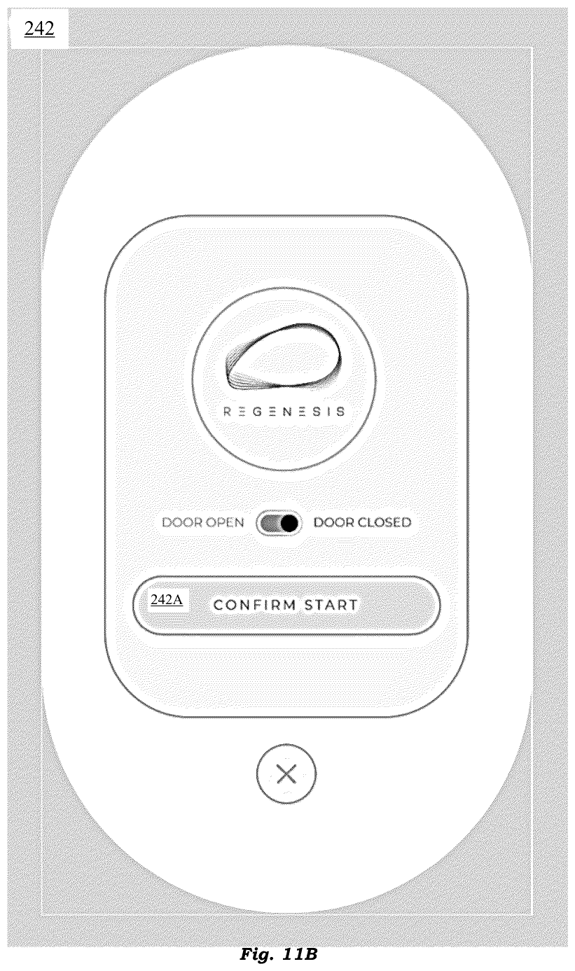

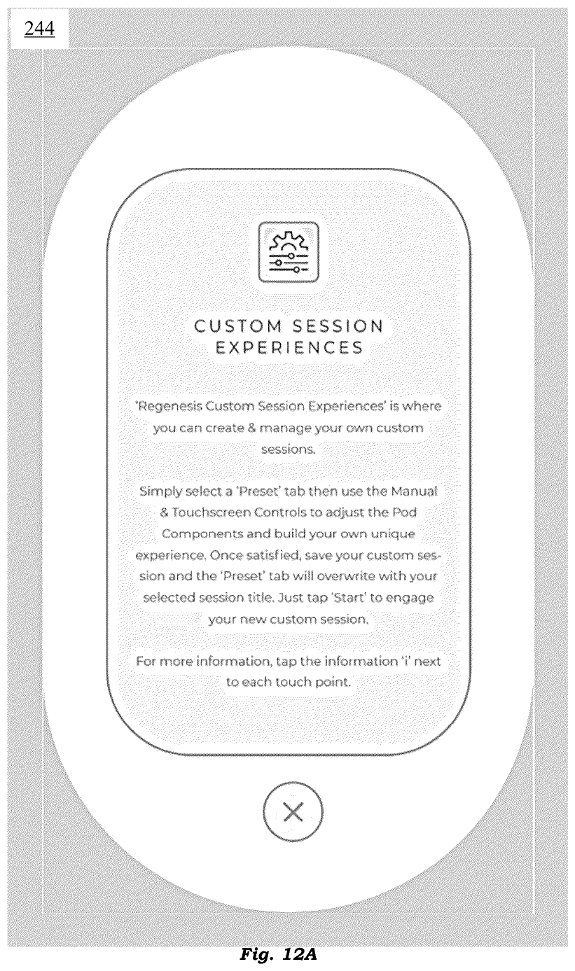

If the user selects or presses or touches Recharge on the screen of FIG. 5A then screen 220 of FIG. 6A is shown on display 148A. Recharge sub-mode is a configuration preset that aims to “recharges” the energy, consciousness and the sense of participation of the user in the multisensory experience. As shown on screen 220, the user can configure various parameters for their recharge session by the GUI widgets shown on the screen. Once done, they can then press Start Session button 220A.

After pressing Start Session on screen 220, the user is navigated to confirmation screen 222 of FIG. 6B for the recharge session, where they can control the duration of the session and still other parameters of the recharge experience. More specifically, the user can select whether door 110 of pod 100 will stay open or closed during the session and whether the user will experience an inner journey or an outer journey. Exemplarily, inner journey employs or activates stroboscopic light source 124 while outer journey does not. Now, the user can select Confirm Start button 222A to commence their recharge session or experience.

In a similar manner, user 150 via touch-sensitive display 148A of pod 100 of FIG. 3 may select any of the other sub-modes i.e. Reconnect 216C, Rejuvenate 216D, Relax 216E and Recover 216F from screen 216. In response, the user is navigated to respective screens 224, 228, 232 and 236 of FIG. 7A, FIG. 8A, FIG. 9A and FIG. 10A respectively. From these screens the user can configure the various parameters of their session and start the sessions of sub-modes 216C, 216D, 216E and 216F by pressing Start Buttons 224A, 228A, 232A and 236A respectively.

The user is then navigated to respective confirmation screens 226, 230, 234 and 238 of FIG. 7B, FIG. 8B, FIG. 9B and FIG. 10B corresponding to sub-modes 216C, 216D, 216E and 216F where they can configure the duration of their session, whether door 110 of pod 100 will stay open or closed during the session and whether they want to experience inner journey or outer journey per above. User 150 can then commence the session by pressing respective Confirm Start buttons 226A, 230A, 234A and 238A of Reconnect sub-mode 216C, Rejuvenate sub-mode 216D, Relax sub-mode 216E and Recover sub-mode 216F.

These sub-modes are named to help users intuitively select experiences based on how they may make them feel. Each mode is designed to guide the user through varying states of stimulation, relaxation, and sensory exploration. Let us look at the rational behind these session sub-modes in greater detail.
1. Recharge sub-mode 216B is a stimulating and upregulating experience, designed to energize the user and enhance alertness.
2. Reconnect sub-mode 216C serves as a balanced journey between Recharge and Relax, often interpreted as a grounding experience that fosters a sense of inner alignment.
3. Relax sub-mode 216E provides the opposite effect, offering a deeply relaxing and downregulating experience aimed at inducing calm and tranquility.
4. Recover and Rejuvenate sub-modes 216F and 216D respectively are distinct modes that may be perceived differently by each user. The primary variations arise from subtle adjustments in settings that contribute to unique experiential nuances.

Sub-modes 216 of the present technology ensure that users can select a session that aligns with their desired state of mind and well-being, enhancing personalization within the multisensory experience.

Aside from pre-configured or preset experiences or sessions discussed above, the present design also allows user 150 to fully customize the various parameters or settings of pod 100. FIG. 11A shows configuration 240 screen of such a custom experience/session where the user can configure and test the various parameters of the system that control the multisensory outputs or stimuli. The user can then touch Start Session button 240A to navigate to confirmation screen 242 shown in FIG. 11B.

Exemplarily, on confirmation screen 242 as shown in FIG. 11B, the user can determine whether door 110 of pod 100 will close or stay open during the experience. Instead of or in addition to the digital control of door 110 offered by screen 242, the user is also able to open/close the door via manual controls on console 144. As such, screen 242 of FIG. 11B may not be necessary depending on the embodiment. Regardless, the user can then commence their session by pressing the Confirm Start button 242A shown in FIG. 11B.

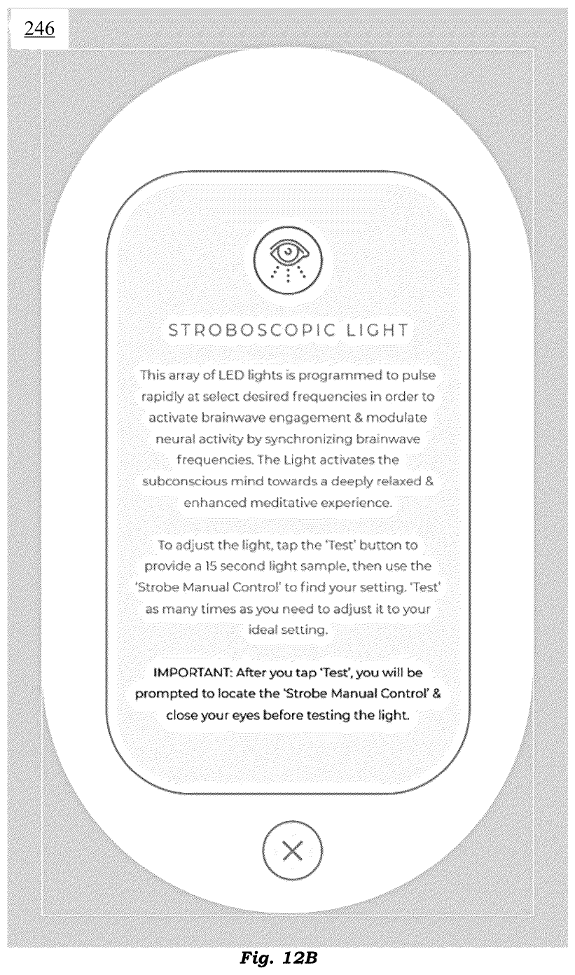

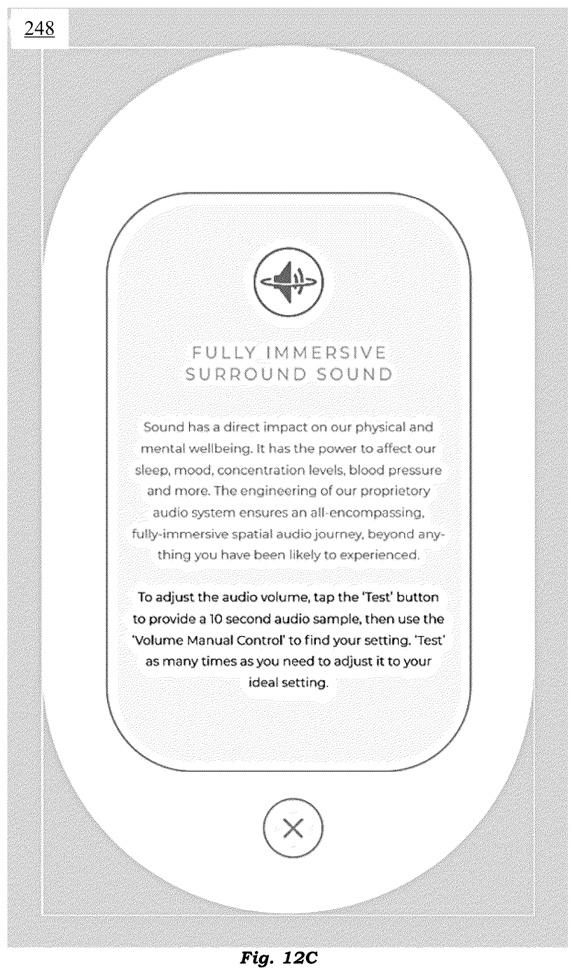

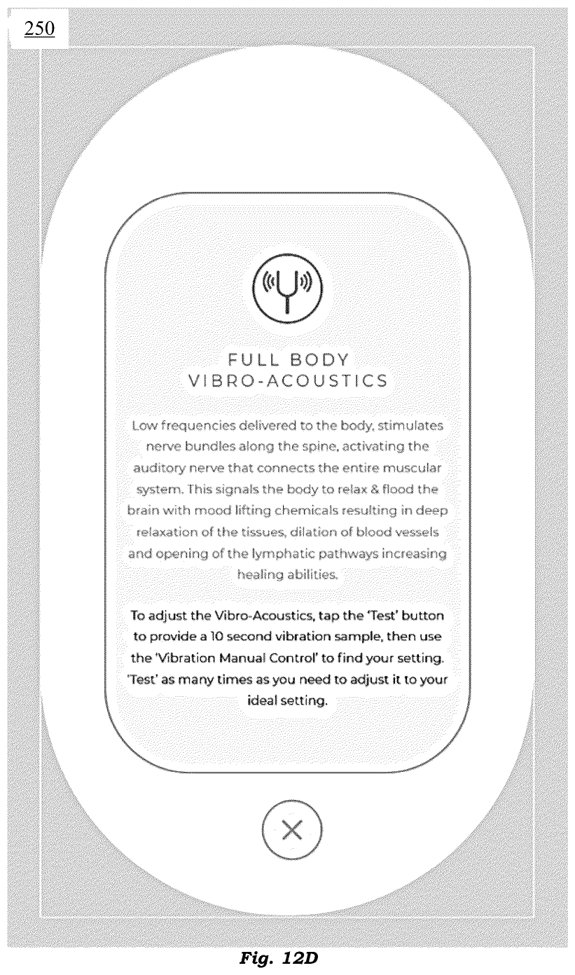

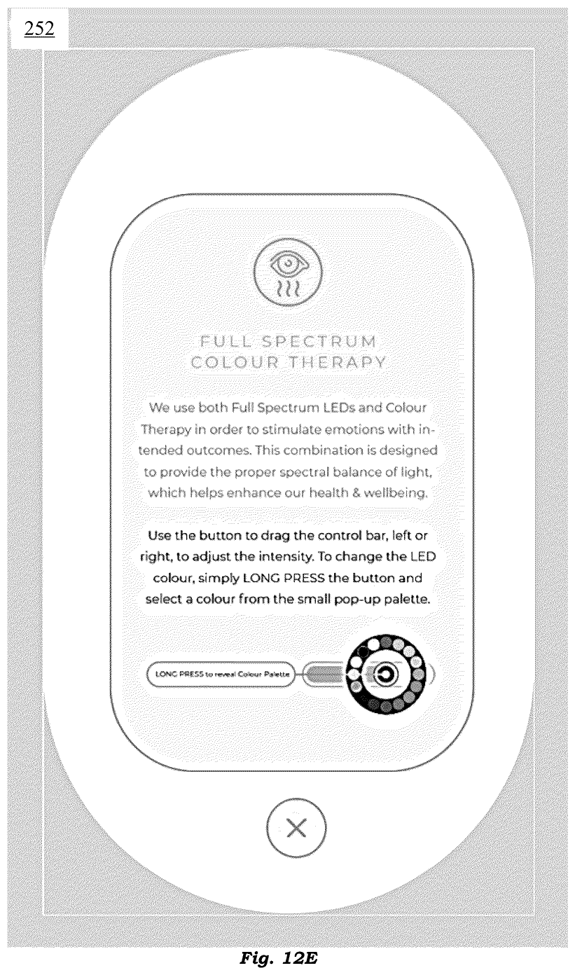

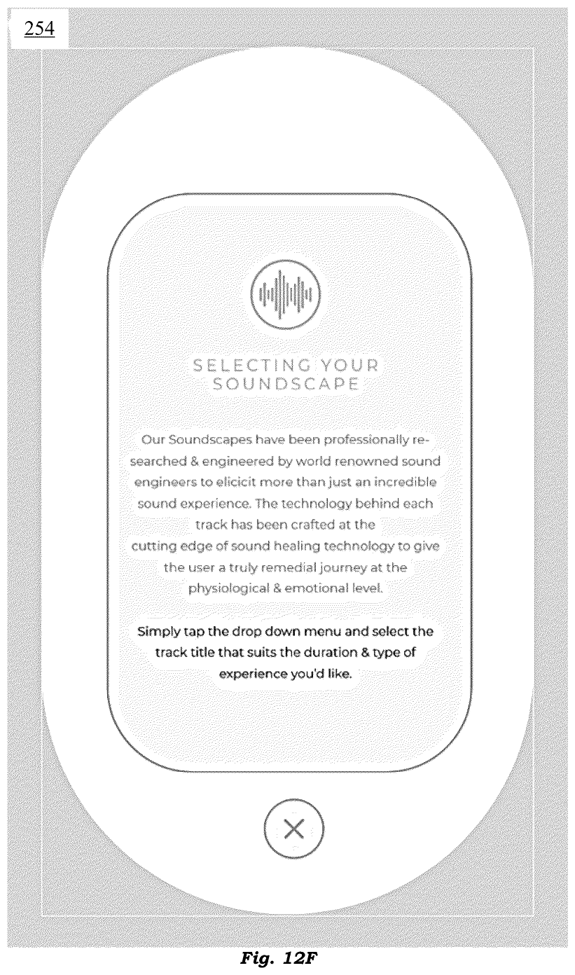

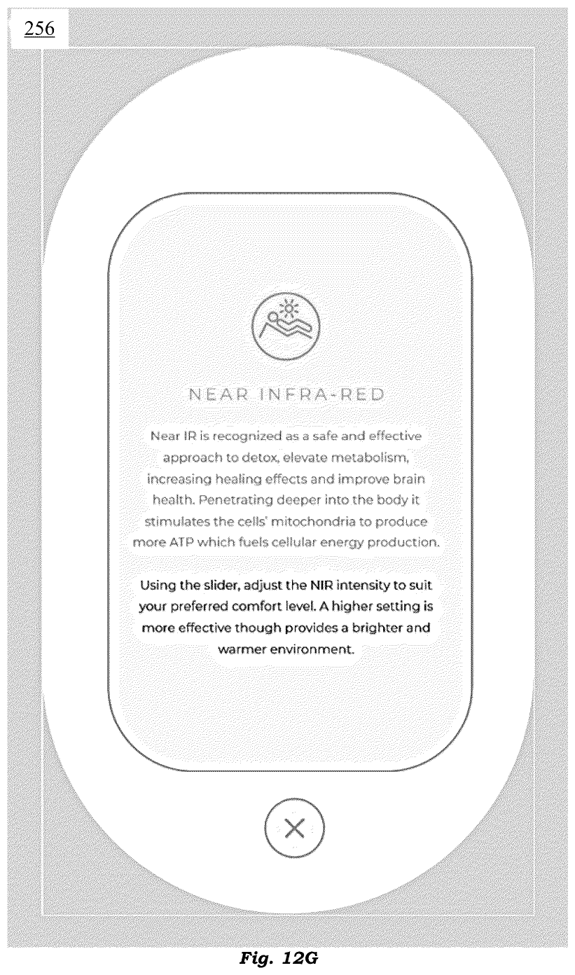

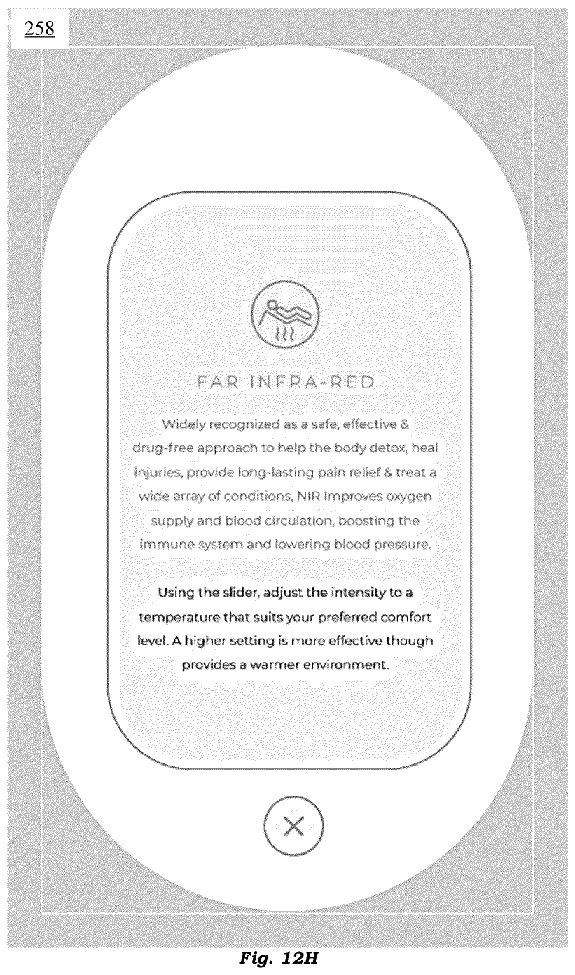

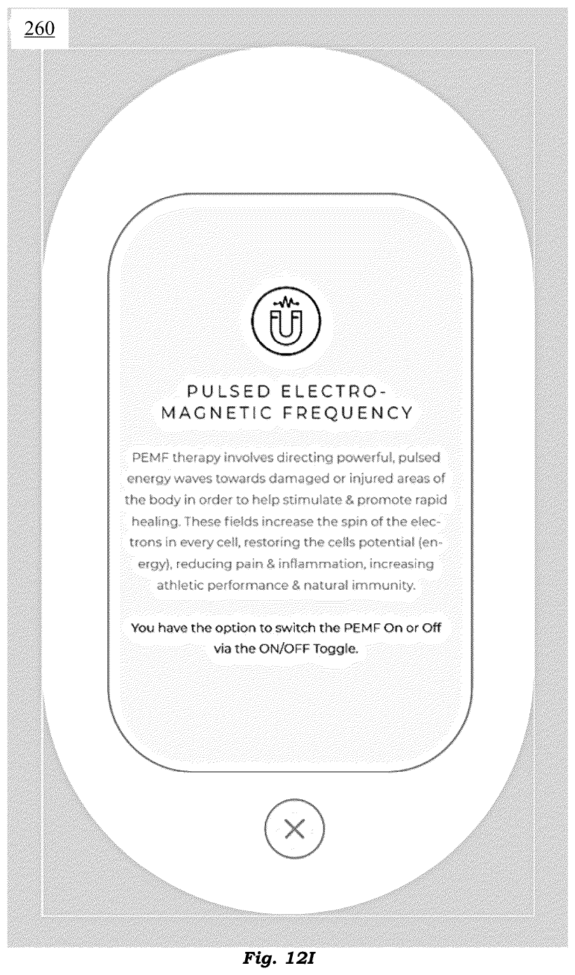

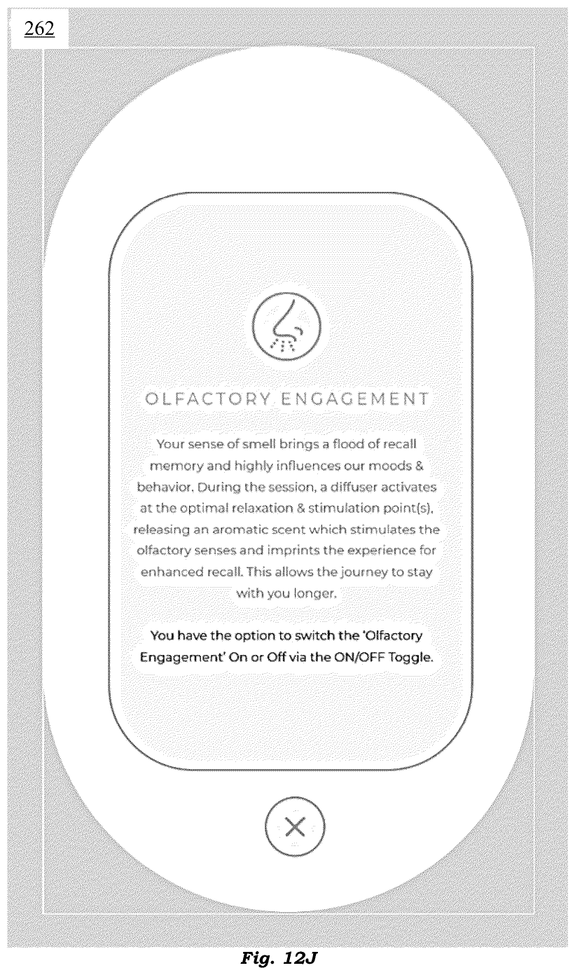

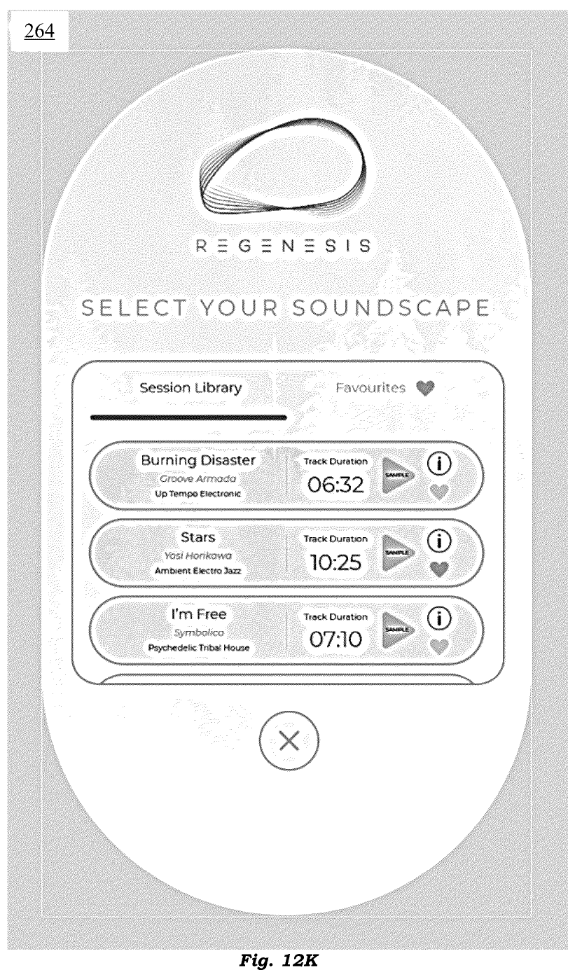

Screens 244-262 of FIG. 12A-J respectively show informational popups explaining custom session and the multisensory outputs or stimuli produced by the system. Each of these popups is displayed when user 150 presses the “i” icon on the configuration screen associated with the specific session or stimulus/output. In the preferred embodiment, the “i” icon is context-sensitive and is available to user 150 on the various screens of the various modes and sub-modes of operation discussed above, including preset and custom sessions/experiences. FIG. 12K shows screen 264 where the user can select an audio or music track or piece for the session from an audio or soundtrack library.

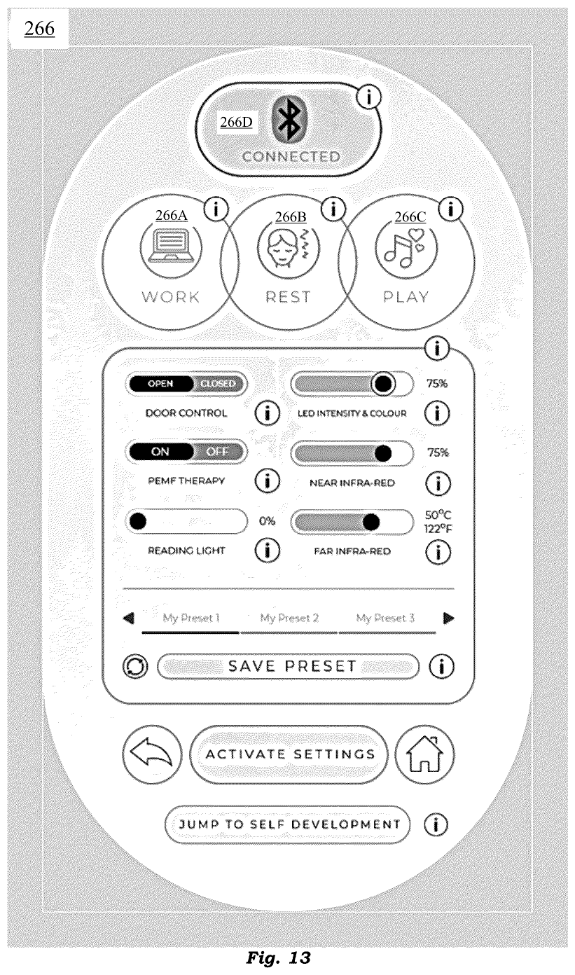

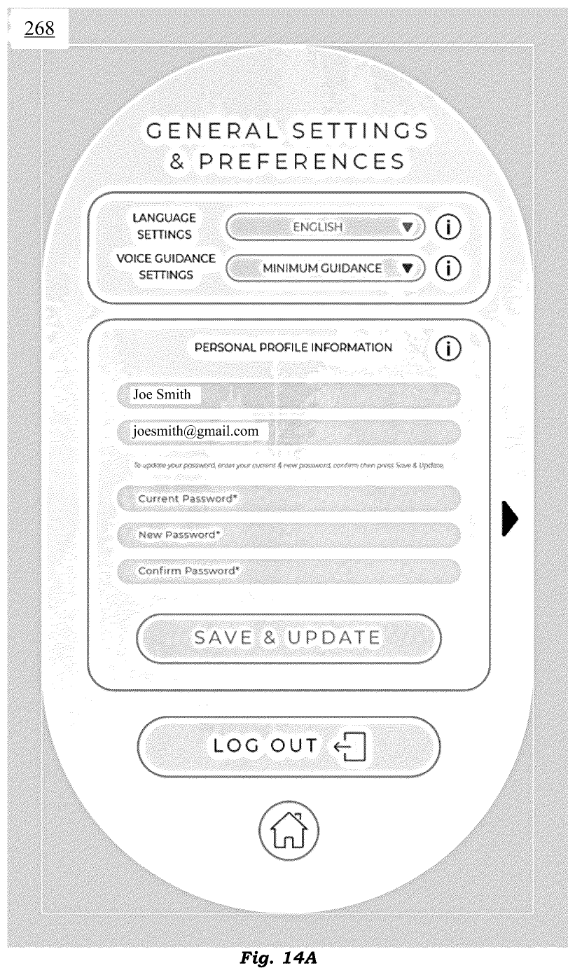

FIG. 13 shows screen 266 for setting and saving the configuration of the Work Rest Play mode of operation of the system introduced above. The Work Rest Play experience is composed of Work, Rest and Play components as indicated by respective icons 266A, 266B and 266C. Notice icon 266D on screen 266 indicating a Bluetooth connectivity of the system to a Bluetooth device carried by user 150. Once connected to the user device, the user can now play audio content from their Bluetooth-connected device to speakers 120. The audio content played by the user may consist of, comprise or be a part of audio performance 122 in various embodiments. The connection to Bluetooth device of the user is performed by pairing the system to the device and by following respective screens on display 148A. Those screens are not explicitly shown in the drawing figures for brevity.

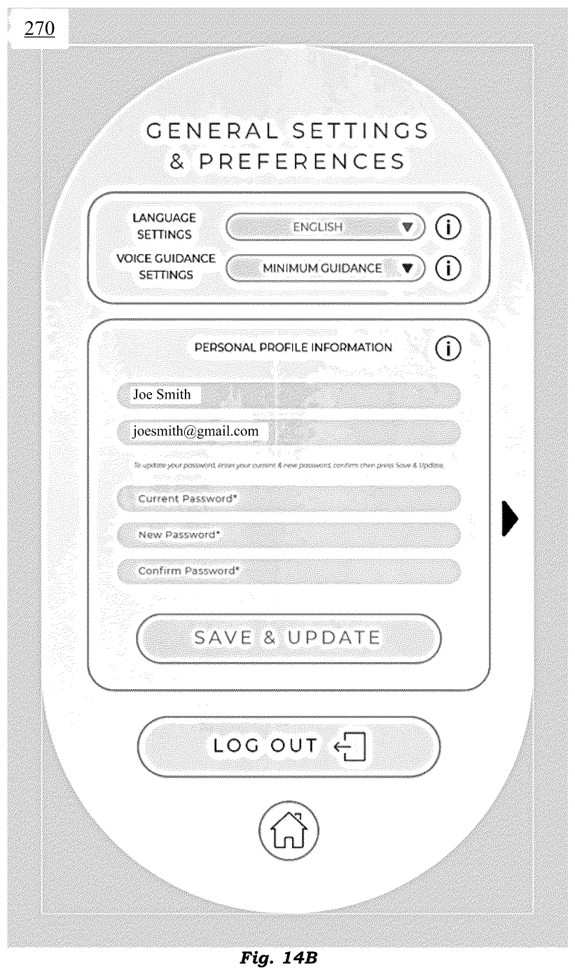

There are also other configuration screens in the system not explicitly shown in the drawing figures that are available to the user for tailoring their multisensory experience in order to optimize or maximize their sense of participation in it. Two such settings and preferences screens 268 and 270 are shown in FIG. 14A and FIG. 14B respectively. There still other configuration screens available to a technician for configuring the overall settings of the system, including the default configurations of the various modes and sub-modes as well as for performing administrative tasks.

The present technology thus integrates/amalgamates a variety of diverse stimuli and associated technologies to produce tailored and immersive sessions/experiences for a variety of users. Since this integration is performed in concert with the binaural beat and audio performance as taught above, one can also refer to such integration as synchronization of the various stimuli and technologies. The various synchronized stimuli generated by an instant for user 150 shown in FIG. 3 include:
1. Audio beat frequency and audio performance 122 generated by an audio system of pod 100.
2. Full spectrum stroboscopic pattern 126 generated by stroboscopic light source 124, and depending on the embodiment, with or without UV LEDS and with or without near-IR LEDs 134.
3. Vibrations 130 produced by vibro-tactile transducers 128.
4. Pulsed low-frequency electromagnetic waves generated by PEMF device/unit 132.
5. Aromas delivered by an olfactory diffuser 142.
6. Electromagnetic waves at frequencies matching Schumann resonances-generated by a Schumann Resonance Generator containing all necessary components including a printed circuit board (PCB) coil. Such a Schumann Resonance Generator is not explicitly shown in the drawing figures but is presumed to exist in certain embodiments.
7. Technology design with emphasis on energetic biogeometric designs and geometric forms.

In the preferred embodiment, the audio system for producing the binaural beat and audio performance 122 is custom tuned to the dimensions of pod 100. Such customized tuning of the audio system is optimized for the position of the head of user 150 and includes a subwoofer 152 shown in FIG. 3A-D under chair 114. The subwoofer is preferably angled/directed towards user 150. Preferably, the audio system includes an audio amplifier that is synchronized with the binaural beat and the audio performance.

In the same or a related embodiment, vibro-tactile transducers 128 are present at specific positions beneath or underneath chair 114 to deliver the most effective vibrations to user 150. Per above, transducers 128 activate in concert with the audio beat frequency and audio performance 122.

In the same or a related embodiment, there are PEMF coils embedded into mat 154 shown in FIG. 3A under the fabric or cushion layer of chair 114 that covers almost the entire length of the chair except the portion where the feet and head of user 150 would rest. Such an arrangement provides the most effective stimulus of pulsed electromagnetic waves by PEMF unit 132 to user 150.

In the same or a related embodiment, mat 154 is also endowed with far infrared (far-IR) heating elements. Advantageously, there is an additional layer of amethyst crystals above the far-IR elements in mat 154. Amethyst crystals naturally emit far-IR wavelengths, so placing these above the far-IR elements elongates the wavelengths and allows them to penetrate farther and deeper into the body of user 150. Such a design results in a more effective far-IR stimulus for the user in the overall multisensory experience.

As shown in FIG. 3E, there are near-IR LEDs arranged in an array 134 of one or more panels behind the head of the user. In the preferred embodiment, there are three panels of near-IR LEDs as shown. The near-IR LEDs utilize a mix of different near-IR and IR LEDs emitting light at different wavelengths. These are preferably curved in arrangement and positioned behind the head of user 150 with a focal area towards the top of the head as shown in FIG. 3E-F and FIG. 3I.

Transcranial near-IR light transmission or low-level light therapy (LLLT) has been proven to be effective at maintaining healthy brain states, improving memory and learning ability, oxygenation due to better blood flow, tissue repair, cell communication, better pain management, reduction of inflammation, neuroplasticity and neuroprotective effects, brain injury, disorder recovery and mood enhancement. The present design provides this stimulus or output to user 150 in conjunction with the other stimuli per present teachings. This leads to stimulation of the brain into a more receptive state, allowing the user to immerse or drop into deeper and more profound multisensory experiences.

As discussed above, there are preferably UV-A & UV-B LEDS positioned above user 150 and alongside stroboscopic light source 124 for the broadest exposure. These important wavelengths of (invisible) light are very valuable to the human body, and yet are some of the most ‘blocked’ wavelengths from us. The present design adds these wavelengths to the full spectrum of light already spanned by the LEDs of stroboscopic light source 124. Per above teachings, the UV LEDs can be configured/programmed to operate at specific times during the various modes and operations of the system.

There also preferably UV-C LEDs contained in pod 100. Preferably, these are positioned at specific angles to broadcast the greatest amount of UV-C exposure to the inside of pod 100. Specific wavelengths of UV-C light are used to sanitize the surfaces of and to create ozone (03) in interior 112 of pod chamber 100 discussed above in reference to FIG. 3.

The UV-C LEDs are only activated during a “sanitization session” between uses, when no human is in pod 100 and the door 110 is closed. The present technology uses load sensing mechanism 146 discussed above to ensure that no one is on chair 114 of pod 100 before the UV-C LEDs are activated. Furthermore, the ozone also acts as a ‘binding/clearing’ agent to help eliminate any remaining odor in pod from the previous session.

Explained further, the residual scent from olfactory diffuser 142 may have a slight lingering smell. However, between user sessions, as pod 100 undergoes a sanitization program, ozone is created within pod 100. Then, one or more exhaust fans 158 shown in FIG. 3A are run during the sanitization program to extract out any remaining smells.

In another preferred embodiment, there is also a Schumann Resonance Generator. This is a PCB antenna coil that emits the frequency 7.83 Hz which is known as the Schumann Resonance frequency. This frequency sits in the Theta wave state and has been shown to provide deep states of relaxation. The Schumann Resonance Generator is not explicitly shown in the drawing figures for brevity but is presumed to exist in such a preferred embodiment.

In the same or a related embodiment, design principles inspired by the science of BioGeometry are used to enhance the environment within pod 100 and to support the bioenergetic fields of user 150.

To summarize, the main distinguishing features of the present technology include:
1. Multi-sensory coherent frequencies-the present design delivers specific frequencies across multiple sensory layers, aiming to explore the emergence of a synergistic and coherent effect in the user. Such an effect creates an optimal state for the user to shift into deep states of relaxation, and to experience great physical benefits.
2. The synchronization of both hemispheres of the brain brings about a balanced state of brain functions, providing the conditions for expanded conscious states to arise.
3. The physiological effects accrued by the systems and methods of the present technology can be utilized to enhance problem-solving skills, boost creative thinking, cultivate the capacity for high-achievement tasks, enhance the ability to perceive interpersonal situations from various angles, and to promote a clearer understanding previously obscured by constant mental distractions within one's own perspective of existence.
4. Stimulating the “mind's eye” of the user with stroboscopic lights shifts their mind into states of deep relaxation and meditation. Simultaneously, they are also exposed to sound frequencies-via the speakers (auditory) and also via the transducers (bodily).
5. Sensory Coherence-The present technology uses a fully immersive multisensory pod environment 100 discussed above to deliver a multi-layered approach, targeting all the senses. This leads to a ‘coherent effect’ on the body and a better sense of participation in the multisensory experience.
6. User feedback-There are feedback mechanisms built into the design to validate the effectiveness of the custom-designed experiences. In other words, user 150 provides an accurate and meaningful feedback as they configure and customize their session via various GUI screens taught above. Per above, these screens and configuration options are available on touch-sensitive display 148A and manual console 144 of FIG. 3.
7. Further, per above teachings, the user enters the pod and selects the session to deliver the experience they want. This may span from full relaxation to ultra-stimulating and anything in between. User 150 is aware of their internal landscape before, during and after the session. Sessions can be customized to an individual. Closing of the door provides a private, comfortable and safe space or environment for the user. They are instructed to relax and close their eyes. The user's hand rests on manual controls or console 144 to allow for immediate adjustment to the intensity of the three key sensory devices (stroboscopic light 124, audio speakers 116 and 120 and vibro-tactile transducers 128).
8. User 150 is exposed to the different healing elements and beneficial frequencies. As user 150 encounters fresh experiences, their mind forges new neural pathways in the brain, leading to profound shifts in consciousness as their brainwaves transition into altered states. At the conclusion of a session, the user is frequently intrigued by these novel experiences, sparking their curiosity about the blend of technologies utilized and the information they have just acquired.
9. While each stimulus or technology is recognized in providing benefits in a standalone manner, the present design innovatively and simultaneously combines or ‘layers’ these otherwise disparate technologies to create ‘Compounding Synergetics’. The result is a powerful cumulative effect that leads to unmatched results.
10. In accordance with user-defined configuration in the preferred embodiment, the same coherent frequencies are preferably used across multiple devices or stimuli. In such an embodiment, at substantially 40 Hz, all of audio beat frequency or the binaural beat from speakers 116, vibro-tactile transducers 128, stroboscopic lights from source 124 and PEMF waves via mat 154 are delivered to user 150.

The key advantages of the present technology include:
1. Increased relaxation of mind and body of user 150.
2. Depending on the session chosen by the user-the mind can be shifted into a downregulated/relaxed state, or upregulating/stimulated state.
3. Increased physiological benefits such as improved blood flow, increase in core body temperature which can provide pain relief and better recovery.
4. Structural, acoustic, emotional and biological resonance.
5. Visually: Stroboscopic light pulses or pattern 126 creates amazing visual effects, focusing attention away from daily chatter of the mind and bringing about brainwave alteration.
6. Audibly: Binaural beat contained within audio/music performance 122 comfortably soothes and entrains the brain, altering brainwave states. Per above, the audio performance 122 is delivered via speakers 120 on either side of the head and via subwoofer 152 positioned below the user.
7. Bodily: Acoustic vibrations from transducers 128 stimulate the limbs and torso of user 150 in harmonic, rhythmic musical frequencies that are connected with the audible sound. Far-IR endowed mat 152 coupled with Amethyst crystals provides another healing element to the experience per above. Amethyst crystals naturally emit far-IR waves, so adding a layer on top of far-IR mat 152 elongates the wavelength, allowing for a deeper penetration of these beneficial waves into the body of user 150.
8. Photoically: Full-spectrum stroboscopic LEDs 124, as well as UV-A & UV-B LEDs bathe user 150 in highly beneficial wavelengths of light.
9. Olfactorily: essential oil fragrances are delivered in bursts to enhance a powerful memory of the experience in the brain of user 150, so that it can be easily recreated, at least in part, after the experience.
10. Endocrinologically: stimulation of brain neurochemical/neurotransmitter production (endorphins, serotonin, dopamine, oxytocin, DMT), bringing states of balance and well-being and enhancing the full experience.

The instant technology is endowed with networking and computer system capabilities that connects and controls the entire equipment and which is easily operated via the intuitive GUI/UI of the above teachings. Per above, there are multiple preset sessions or experiences with tweaks in the settings for each session. The system further allows the user to fully customize a session based on their preferences.

In fact, depending on the embodiments, the fully customizable design of the present technology allows user 150 to benefit from the variety of stimuli offered by pod 100. It does so with or without those stimuli being in concert with the audio beat frequency or binaural beats, and with or without those stimuli being in concert with the audio performance 122 of the above teachings.

Further, there are manual controls available via console 144 that allow the user to adjust the intensity level of the experience. From these, user 150 can control:
1. The brightness of stroboscopic lights 124.
2. Audio volume.
3. Intensity of vibrations from transducers 128.

These levels can be adjusted at any time during the session. At any stage during the session, the user can also pause/stop the session and then make further adjustments to their settings.

Furthermore, prior art systems having an open physical design do not provide the privacy necessary for a user to fully relax and be vulnerable. In such open systems, users remain exposed to external interruptions, ambient light, and noise, which can prevent deep immersion in the experience. In contrast, the present technology offers a fully enclosed environment within pod 100, with door 110 closed, creating a private, safe, and comfortable space. This allows users to truly relax and immerse themselves in deeper states without external distractions.

In contrast, by providing the user a private enclosure of pod 100 with its door 110 closed, the present technology gives the user a private, safe and comfortable environment to immerse into deeper states.

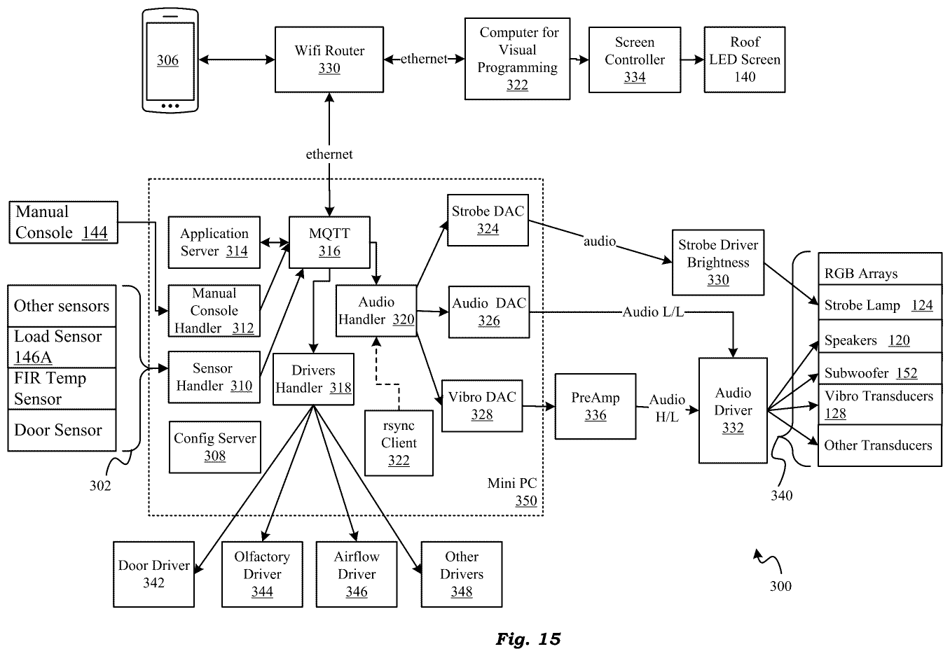

Let us now review the computer design and architectural aspects of the present technology. FIG. 15 presents an overall technical computer architecture 300 of pod 100 of the above teachings. For the following discussion focusing on computer hardware and software technologies of an instant pod 100, we may also simply refer to pod 100 by its computer architecture 300 for brevity. In FIG. 15, the various sensors present in the pod are denoted by right brace 302. The outputs of these sensors are processed by a sensor handler computer program 310 running on a mini personal computer (mini-PC) 350 installed in the interior 112 of pod 100 in a manner so as not to interfere with the aesthetics of the pod for user 150.

The various modules of pod system or pod 300 use publish/subscribe scheme of message-based communication that preferably employs Message Queuing Telemetry Transport (MQTT) 316 as shown in FIG. 15. There is also an application server 314 running on mini-PC 350 that processes and interprets the MOTT messages. There is a manual console server 312 operating on mini-PC 350 that is in charge of controlling manual console 144 of the above teachings. There is also a drivers handler program 318 that is in charge of controlling the various device drivers in pod 100/300, including door driver 342, olfactory driver 344 and airflow driver 326 explicitly shown in FIG. 15, as well as any other drivers 348.

There is an audio handler program 320 that is responsible for driving the various transducers of the pod in concert with the binaural beat and audio performance 122 of FIG. 3. More specifically, audio handler 318 drives a strobe digital-to-analog converter (DAC) 324 that controls the brightness of stroboscopic lights or lamps 124 via a strobe brightness driver 330. Handler 320 also controls audio driver 332 responsible for driving speakers 120 and subwoofer 152.

Audio handler 320 also controls a vibro DAC 328 that is responsible for controlling vibro-tactile transducers 128 of the above teachings via a pre-amplifier 336 and audio driver 332. As a result, the vibrations of transducers 128 are produced in concert with audio beat frequency and audio performance 122 of the prior teachings. In this manner, the various transducers present in pod 300 as shown by left brace 340 are driven/controlled by the respective drivers of the instant system. Thus, the system receives inputs from sensors 302 on the left and ultimately transmits outputs to or drives the various transducers 340 on the right by utilizing a number of software and hardware modules as illustrated in FIG. 15 and taught herein.

Mini-PC 350 also runs an rsync client for updating the pod software from a central server via network connectivity afforded by a wifi router 330. There is also configuration server 308 that runs on mini-PC 350 that is responsible for administering the various configurations of the sessions/experiences taught above. There is also a configuration application that a technician can use to configure the system via a configuration app running on device 306 connected to wifi router 330. There is also a workstation/computer 322, preferably Windows PC, running visual programming software for driving roof screen 140 of the above teachings via screen controller 334. Preferably, roof screen 140 is an LED screen as shown in FIG. 15. In an alternative embodiment however, roof screen 140 is an LCD screen. Screen 140 may utilize any suitable display technology within the current scope.

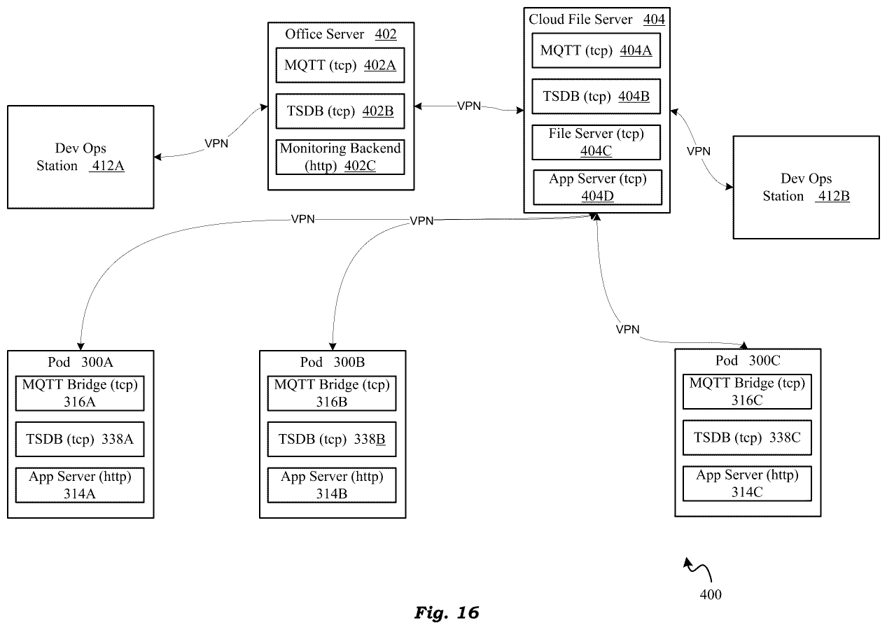

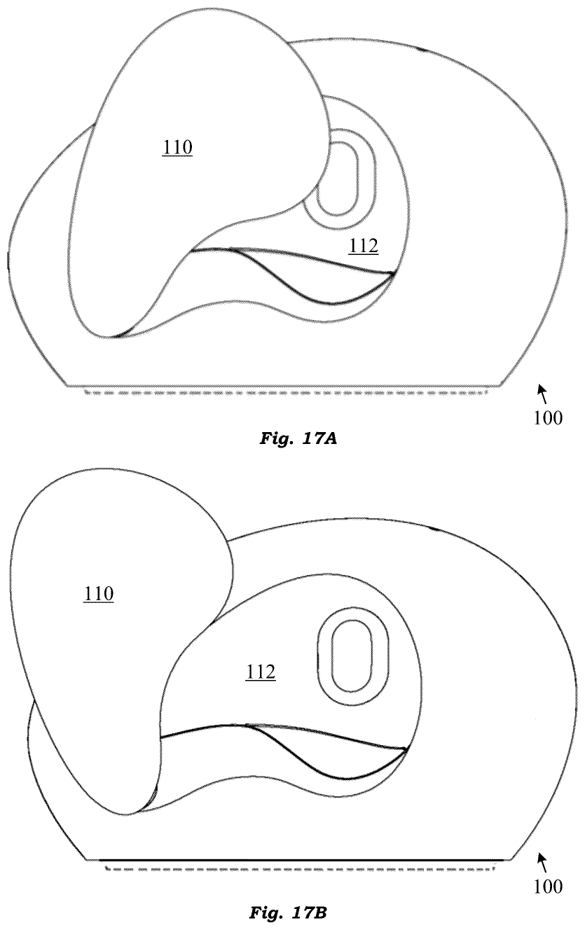

FIG. 16 illustrates network architecture 400 of the present design. As shown in FIG. 16, there is a central file server 404 operating in the cloud. Cloud file server 404 runs a messaging sub-system or module 404A based on MQTT. There is also a time series database (TSDB) 404 executing on server 404. There is also an application server 404D operating on cloud server 404. Cloud server 404 securely communicates via an instant virtual private network (VPN) to an office server 402 operating respective counterparts of the software modules, namely MQTT 402A, TSDB 402B as well as a monitoring backend module 402C. Development Operations (DevOps) systems can connect to either office server 302 or cloud server 404 to develop and maintain software for and to manage the overall operation of a network of instant pods. Two such DevOps stations 412A and 412B are shown in FIG. 16.

As discussed above, various computer modules in an instant pod with a technical architecture 300 discussed above communicate via a pub/sub scheme. Such a scheme utilizes MQTT Bridge or simply MQTT 316 operating in pod 100 and specifically on its mini-PC 350 as discussed above. As such, for three exemplary pods 300A, 300B and 300C shown in FIG. 16 there are respective MOTT bridges 316A, 316B and 316C as shown. There are also respective application servers 314A, 314B and 314C. There are also respective counterparts 328A, 328B and 328C of TSDB 404B of cloud file server 404 that are present in pods 300A, 300B and 300C as shown. Pods 300 also communicate with cloud file server 404 over the instant VPN in a secure manner.

Thus, based on the above-described computer architecture 300 and network design 400, a network of instant pods can be efficiently and securely maintained from a central office and server. Any updates from DevOps systems 412 are securely propagated over the VPN via cloud server 404 and ultimately to pods 100/300 of the present teachings.

Let us now focus on door mechanism 162 introduced earlier in reference to FIG. 3C and which is responsible for the operation of door 110 of pod 100. Door mechanism 162 allows door 110 to open and close, either by manual or electronic means, per above teachings. FIG. 17A shows door 110 of pod 100 in a half open position while exposing its interior 112 and FIG. 17B shows the door in the fully open position. Door mechanism 162 is preferably optimized for a smooth, relaxed and fluid operation of door 110. Let us now study various implementations of door mechanism 162 in detail.

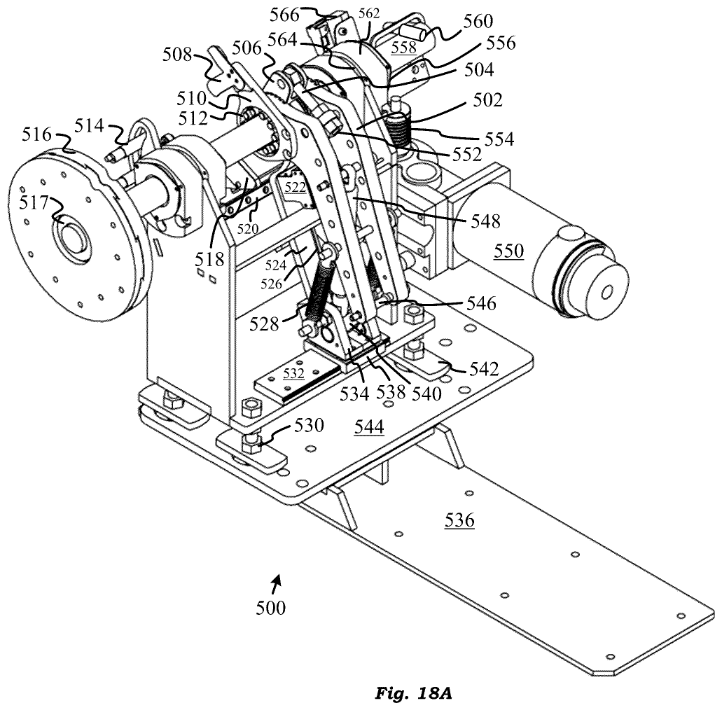

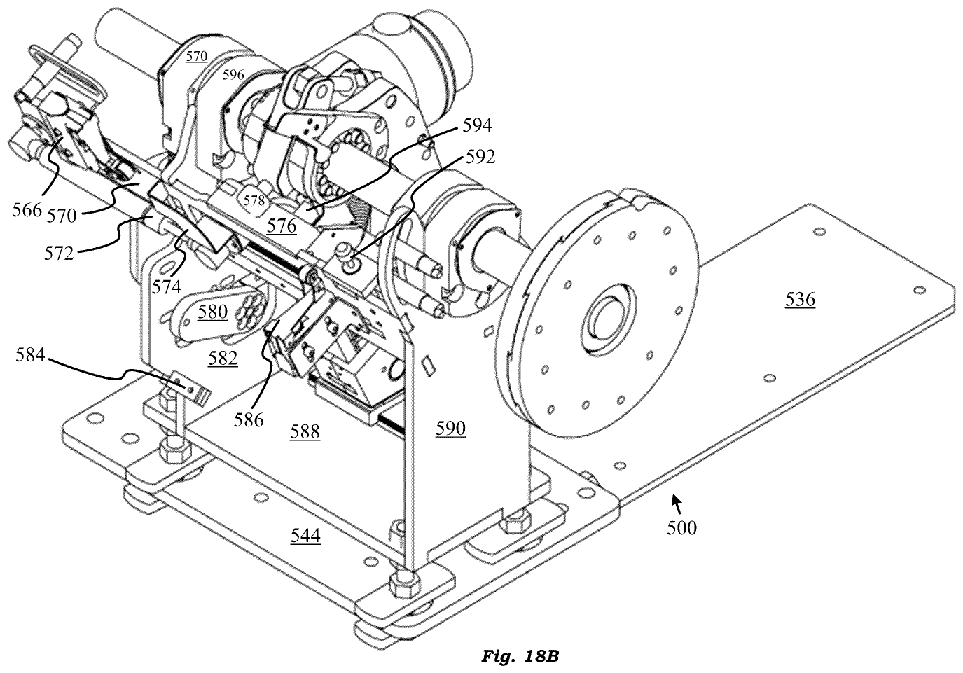

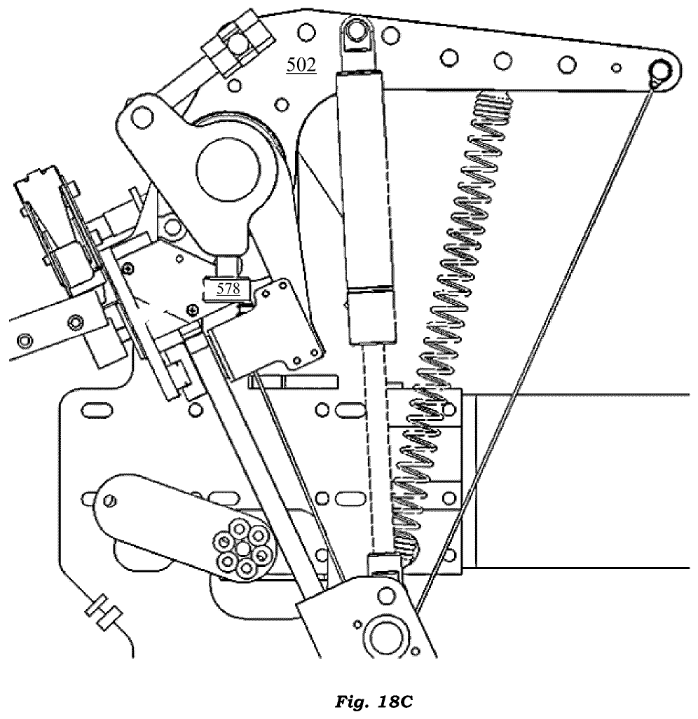

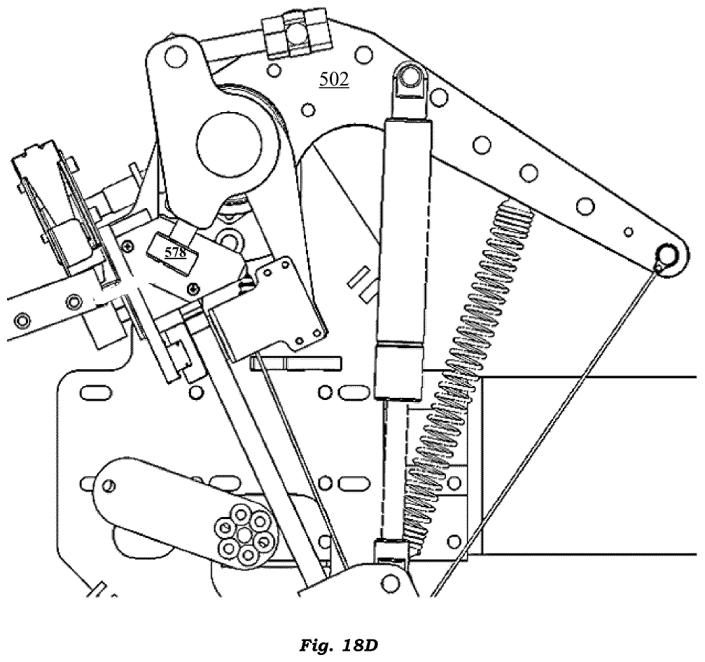

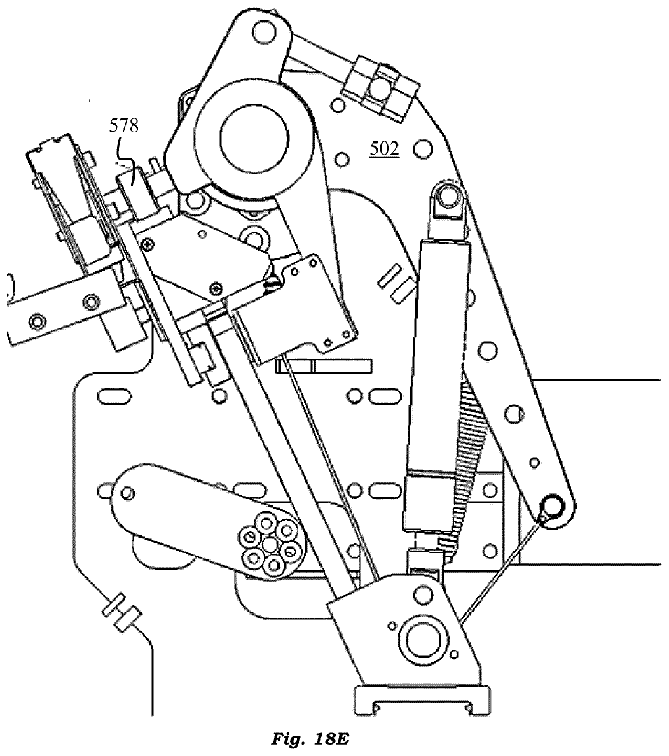

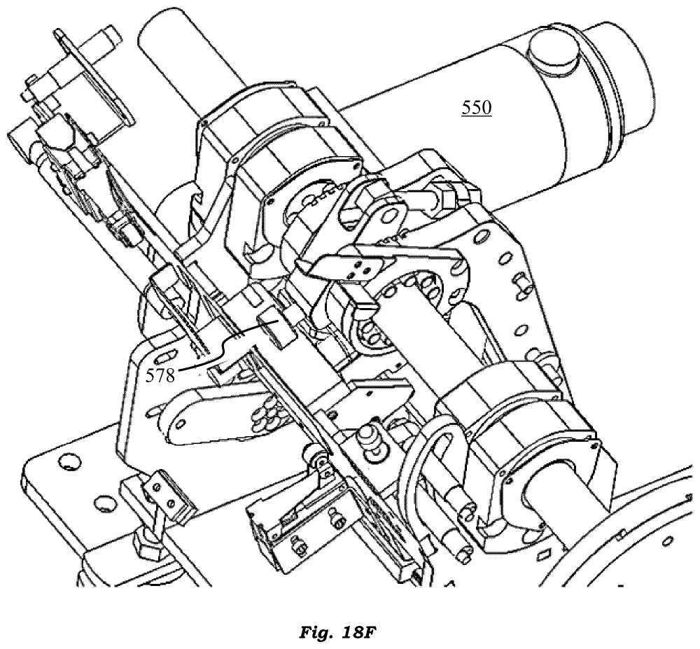

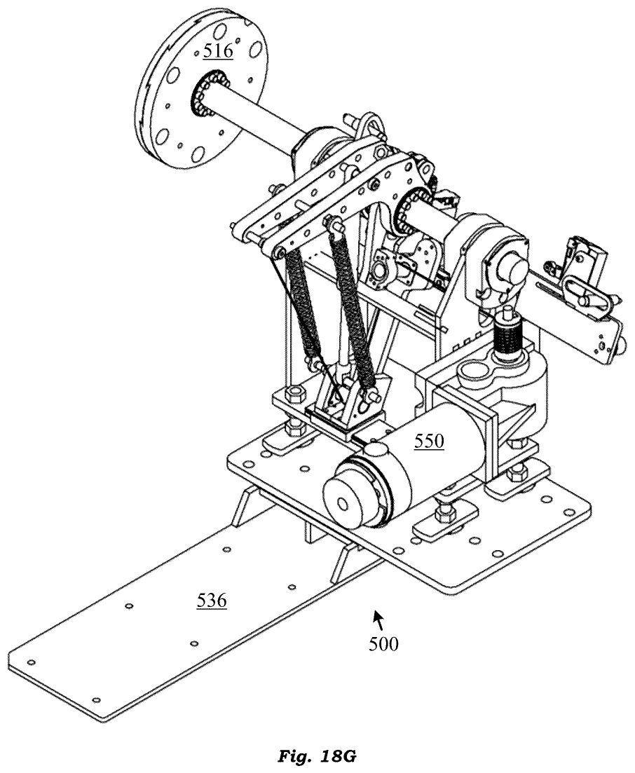

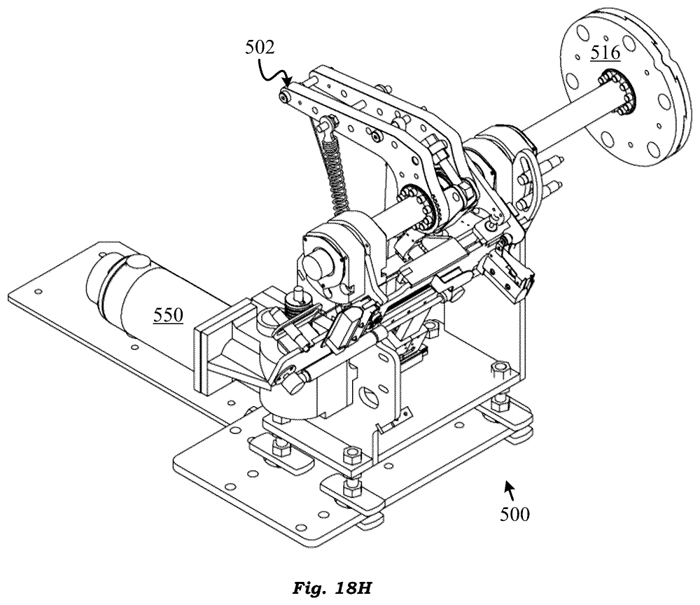

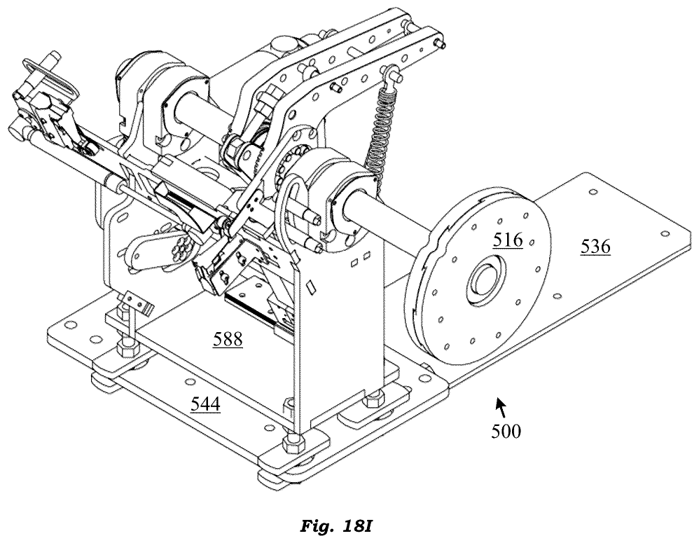

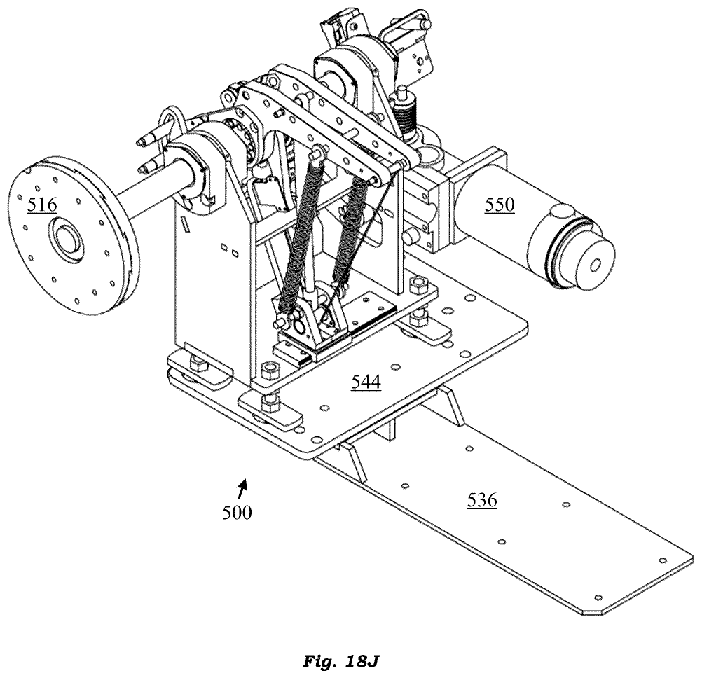

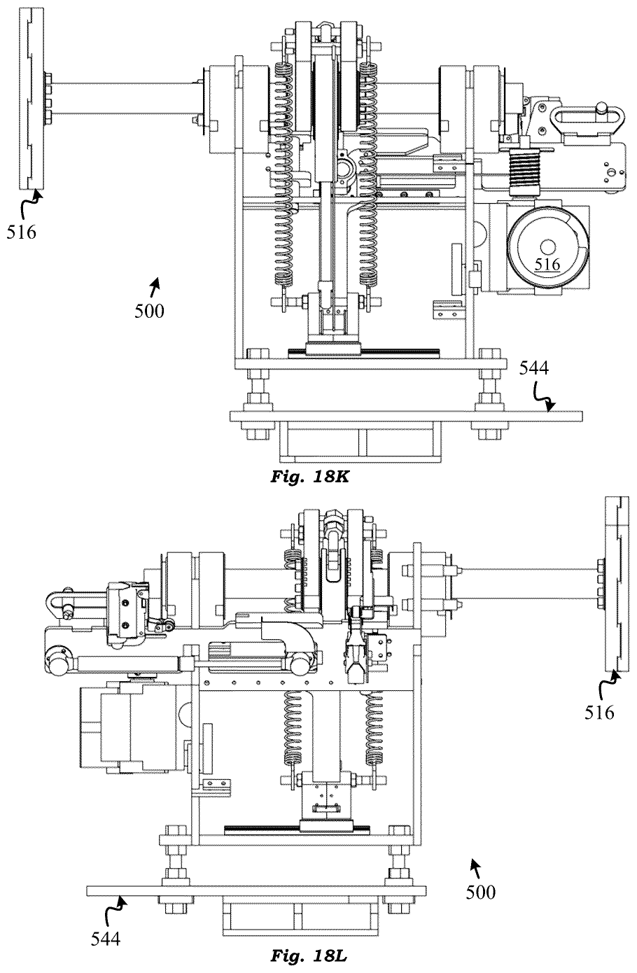

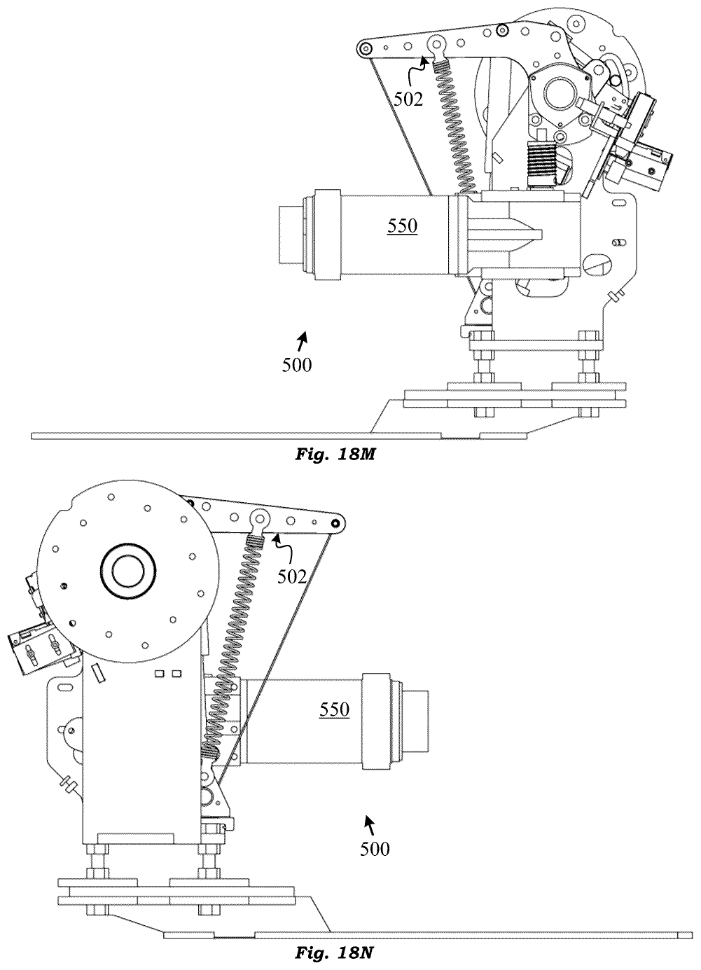

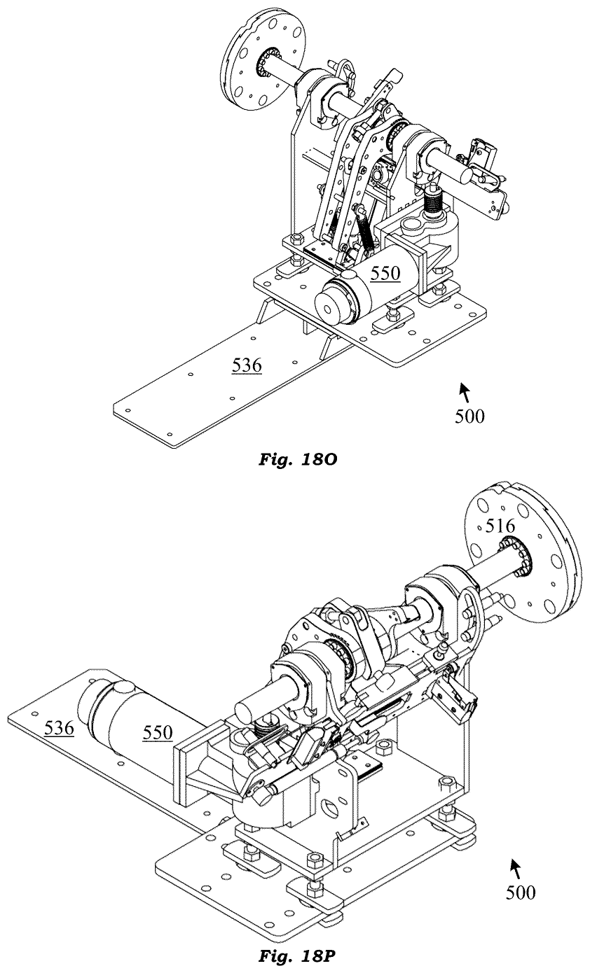

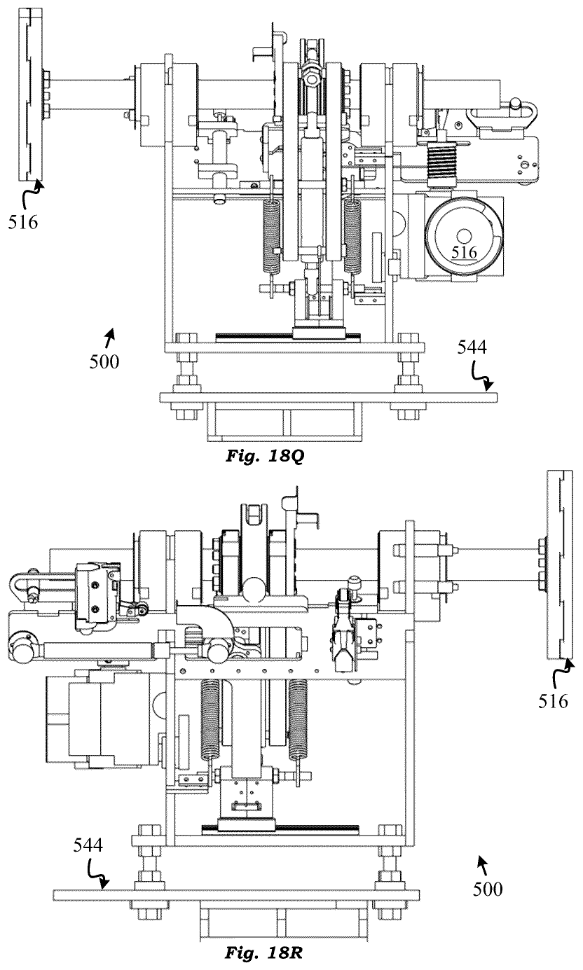

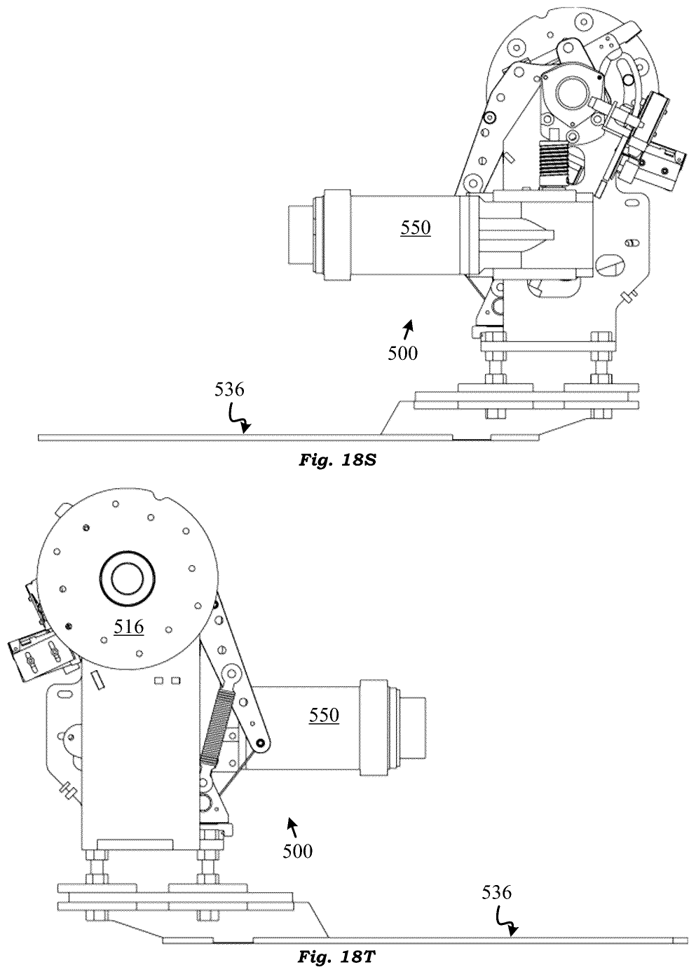

In the preferred embodiment, door mechanism 162 is implemented by an innovative string mechanism 500 as shown in FIG. 18. Such a string-based door mechanism or simply a string door mechanism 500 allows door 110 to be pushed or slid out of pod 100 from its closed position and then be rotated to an open position. FIG. 17A-B show two exemplary open positions of door 110 while FIG. 2C shows it in the fully closed position. FIG. 18A-B show two perspective views of present string door mechanism 500 for powering/operating door 110 of pod 100. The various parts or components of mechanism 500 are shown by the reference numerals in FIG. 18A-B when door 110 is in the fully closed position or condition as shown in FIG. 2C.

In particular, FIG. 18A shows moment arm 502, shaft rotation angle adjuster bolt 504, shaft rotation angle adjuster 506, sensor trigger plate 508, dampener 510, locking arm assembly/power lock 512, proximity sensor 514, door connector plate 516, locking assembly/power lock 517, rotational motion cam follower track 518, linear rail 520, sliding bar connection plate 522, slider connection bar 524, stainless steel string 526, extension spring 528, mounting adjuster bolt 530, linear rail 532, sliding pivot base 534, mounting base plate 536, linear block 538, stainless steel string pivot shaft 540, custom washer 542, adjuster base plate 544, stainless steel string hook 546, gas spring 548, brushed DC motor 550, adjuster nut 552, stainless steel string roller 554, bearing stopper 556, stainless steel shaft 558, proximity sensor 560, shaft bearing holder 562, needle roller bearing 564 and limit switch 566 of present door mechanism 500.

Of the above components, moment arm 502, shaft rotation angle adjuster bolt 504, shaft rotation angle adjuster 506, sensor trigger plate 508, dampener arm 510, door connector plate 516, rotational motion cam follower track 518, sliding bar connection plate 522, slider connection bar 524, sliding pivot base 534, mounting base plate 536, custom washer 542, adjuster base plate 544, stainless steel string hook 546, stainless steel string roller 554, bearing stopper 556 and shaft bearing holder 562 are innovatively custom designed for mechanism 500 of the present technology.

In a similar manner, FIG. 18B shows limit switch 566, rear mounting plate 570, gas spring for sliding motion 572, limit switch trigger plate 574, cam follower sliding motion track 576, cam follower 578, DC motor's clutch lever 580, right wall mounting plate 582, clutch string holder 584, limit switch 586, upper base plate 588, left wall mounting plate 590, shock absorber 592, stainless steel string pivot shaft 594, slider bearing 596 and needle roller bearing 598 of present door mechanism 500.

Of the above components, rear mounting plate 570, limit switch trigger plate 574, cam follower sliding motion track 576, clutch lever 580 of DC motor 550, right wall mounting plate 582, clutch string holder 584, upper base plate 588 and left wall mounting plate 590 are innovatively custom designed for door mechanism 500 of the present technology.

FIG. 18C-D show left-side half section views of door mechanism 500 when door 100 of pod 100 is in the fully open and half open positions/conditions respectively. Notice the position of cam follower 578 from FIG. 18A-B in FIG. 18C-D. Not all the components of mechanism 500 are explicitly marked in FIG. 18C-D to avoid distraction from the main principles behind the design of mechanism 500 being taught.

Similarly, FIG. 18E shows left-side half section view of door mechanism 500 of the present technology while door 110 is sliding in and out of pod 100. FIG. 18F shows left-side half section view and top-front-left corner view of door mechanism 500 when door 110 of pod 100 is in the fully closed position of FIG. 2C. Once again, notice the position of cam follower 578 in FIG. 18E-F. FIG. 18G-J show additional perspective views of mechanism 500 while FIG. 18K-N show front, rear, right, left views respectively of mechanism 500 when door 110 is in the fully open position. For completeness, FIG. 180-P provide additional perspective views while Fig. Q-T provide front, rear, right and left views respectively of door mechanism 500 when door 110 is in the fully closed position. Again, not all the components of mechanism 500 from FIG. 18A-B are explicitly marked in FIG. 18E-T to avoid clutter.

Now, let us look at the operating or working principle of door mechanism 500. Door mechanism 500 is able to work in two conditions, firstly under normal conditions, and secondly in an emergency situation/condition. In normal conditions, there are two operations that can be performed, door opening and door closing, while in emergency conditions, only door opening operation can be performed.

Normal Operation of Door Mechanism 500:

Normal conditions refer to the situation when the power/electricity supply to pod 100 is available, the electronic control is still functioning properly, and there is no damage to the mechanical components of door mechanism 500. Under normal conditions, there are three components that act as actuators for opening and closing of door 110. The first component is DC motor 550, the second is gas spring for translation 572, and the third is gas spring for rotation 548.

In order to perform the opening operation of door 110 with the initial condition of door mechanism 500 fully closed as shown in FIG. 18F, door opening command(s)/instruction(s) is/are sent from the electronic controller. This causes DC motor 550 to rotate and which in turn causes string roller 554 to rotate counter-clockwise, thus unwinding stainless string 526. At the same time, gas spring for translational movement 572, which is in a compressed state, starts to push main stainless shaft 558 through slider connection bar 524 and sliding bar connection plate 522. This causes door 110 to start to open with a linear movement outward. This linear sliding movement is guided by linear rail 532 and linear rail 520.

At the same time, gas spring for rotational movement 548, which is in a compressed state, also provides an upward force to rotate stainless shaft 558 through moment arm 502. However, as long as cam follower 578 is on sliding motion track 576, stainless shaft 558 is not able to rotate so that door 110 is also not allowed to rotate. In this condition, the only movement that can occur is a straight movement outward from the pod.

During the process of unwinding stainless steel string 526, when cam follower 578 reaches rotational motion track 518, stainless steel shaft 558 stops moving linearly and the thrust from rotational gas spring 548 starts to cause rotational motion on shaft 558. This causes door 110 to rotate open upwards. The opening process continues until the trigger plate sensor triggers proximity sensor 514, which causes DC motor 550 to stop. By this time, the opening of door 110 has been completed and successfully.

If proximity sensor 514 does not trigger, this would cause shaft 558 to rotate more than it should. In order to prevent this, there is a limit switch 586 that is triggered by trigger plate 508. This results in the disconnection of power supply to DC motor 550, specially to cut off its rotation in the direction of unwinding stainless steel string 526. With the protection mechanism of limit switch 586, unwinding process does not happen any further. However, the operation for winding string 526 to close door 110 is still permitted and can be carried out.

In order to perform the process of closing door 110 from an initial condition of door mechanism 500 fully open as shown in FIG. 18C, a closing instruction is sent from the electronic control to DC motor 550. This causes string roller 554 to rotate clockwise, thus rolling up stainless string 526. In the rolling process, as long as cam follower 578 is on rotational motion track 518, any linear sliding movement is not possible. This causes string 526 to pull moment arm 502 downward. This in turn causes shaft 558 to rotate and rotationally close door 110. In addition, this also causes moment arm 502 to perform a compression action on rotational gas spring 548.

When cam follower 578 begins to touch sliding motion track 576, the movement of shaft 558 switches from rotation to linear translation inward. This causes door 110 to close linearly with translational s spring 572 being compressed. This linear movement continues until proximity sensor 560 is triggered by shaft 558, thus causing DC motor 550 to stop. By this time, the process of closing of door 110 has been completed successfully.

If proximity sensor 560 does not trigger, this would cause translation of shaft 558 to be more than it should. In order to prevent this, there is a limit switch 566 that is triggered by trigger plate 574. This results in the disconnection of power supply to DC motor 550, specially to cut off its rotation in the direction that would roll up stainless steel string 526. With the protection mechanism of limit switch 566, the rolling process does not happen any further. However, the operation to unwind string 526 to close door 110 is still permitted and can be carried out.

Operation of Door Mechanism 500 in an Emergency:

An emergency condition refers to the situation when there is no power supply to door mechanism 500 and/or its electronic control is damaged, whilst its mechanical components are functioning properly.

In an emergency situation, when door mechanism 500 is fully closed as shown in FIG. 18F, translation gas spring 572 and rotation gas spring 548 are in their compressed state. Under this condition, translation gas spring 572 actively provides an outward thrust to shaft 558, and rotation gas spring 548 actively provides an upward thrust to rotate shaft 558. However, the movement of shaft 558 does not occur, because DC motor 550 cannot be rotated in a back-drive manner through external force. So stainless steel string 526 cannot be rolled or released. This back-driven limitation occurs because of the worm gear box mechanism used in motor 550. The back-driven process or rotation of string roller 554 through external force can only be done if the clutch connecting the output shaft of DC motor 550 with the worm gear box inside, is disengaged or deactivated.

Thus, opening of door 110 during an emergency is done by turning clutch lever 580 to the disengaged position, or by pulling the clutch pulling string connected to clutch lever 580. When clutch DC motor or simply DC motor 550 is deactivated or disengaged, string roller 554 can rotate freely. This causes stainless steel string 526 to be pulled freely by the combination of the thrust provided by translational gas spring 572 and rotational gas spring 548, which were previously in a compressed condition. So, shaft 558 can move linearly and then rotate, which causes door 110 to open by itself, without requiring any movement from DC motor 550.

The opening movement that occurs with this process is more aggressive compared to the opening movement that is carried out normally by involving the rotation of DC motor 550. To minimize the aggressive nature of the opening movement, extension spring 528 and shock absorber 592 serve to absorb some of the thrust that occurs when door 110 is opening in an emergency situation.

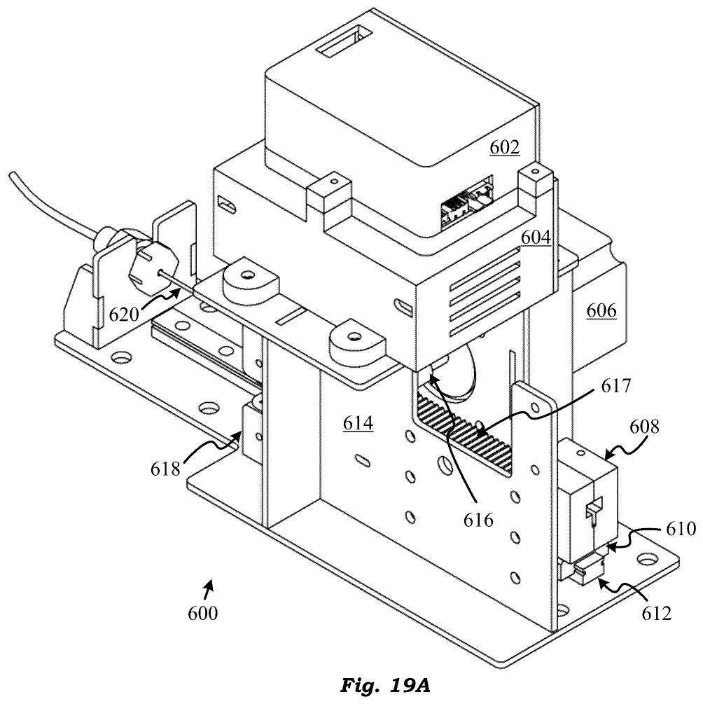

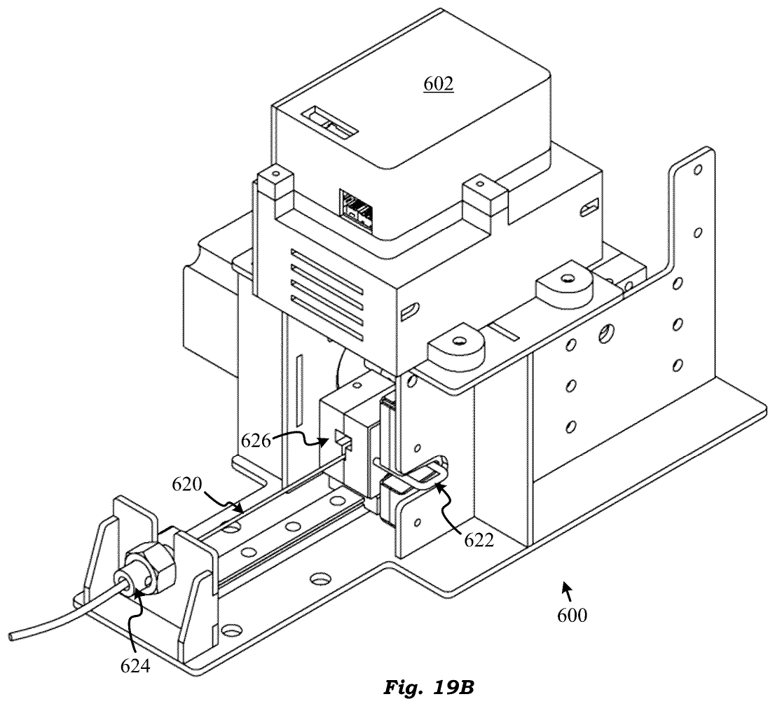

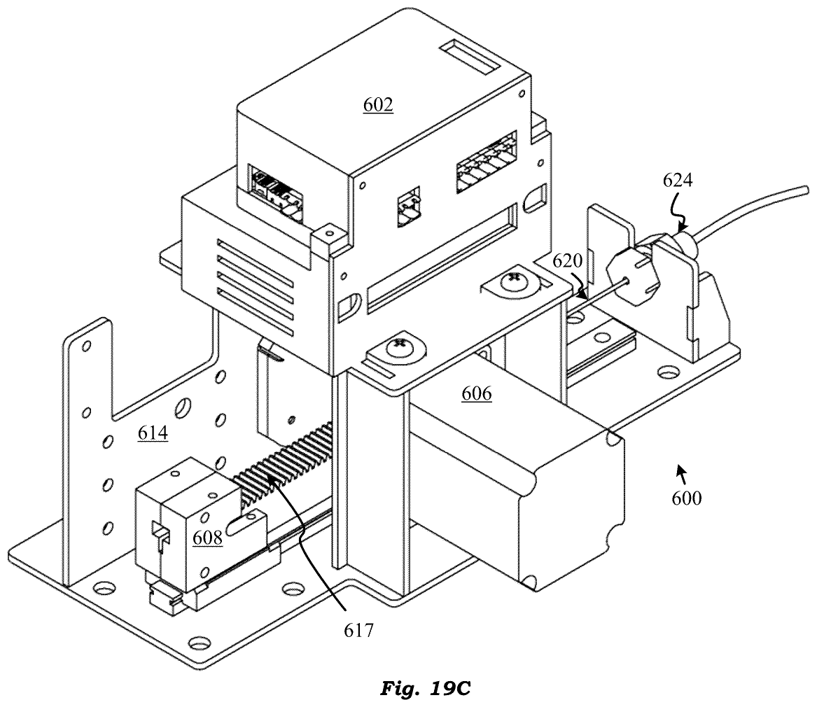

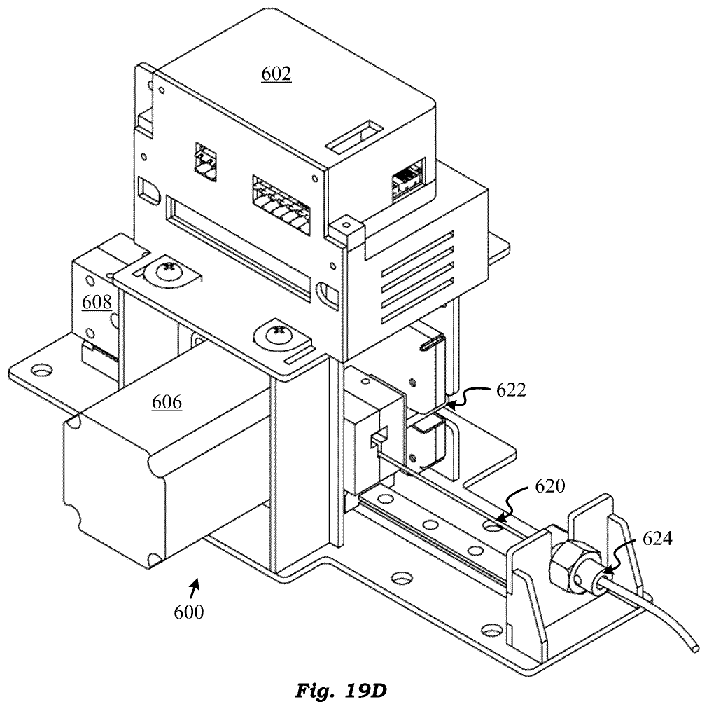

FIG. 19A-D show an automatic release enhancement 600 to door mechanism 500 of FIG. 18A-T for controlling and powering the movements of door 110 of pod 100 of the above teachings. More specifically, mechanism 600 automatically opens door 110 in the case of a power failure. Thus, a string door mechanism 500 equipped with automatic release 600 may be referred to as an automatic release string door mechanism of the instant technology. The perspective views of automatic release 600 of FIG. 19A-B show automatic release controller 602, stepper motor driver 604, stepper motor 606, rack gear holder 608, linear block 610, linear rail 612, metal frame 614, pinion gear 616, rack gear 617, electromagnetic door lock 618, clutch pulling string 620, lock pin 622, clutch pulling string locking bolt 624, and rack holder and pulling string connector 626.

Of the above components, automatic release controller 602, rack gear holder 608, metal frame 614, clutch pulling string locking bolt 624, and rack holder and pulling string connector 626 are innovatively custom designed for automatic release 600 of the present technology. For completeness, FIG. 19C-D provide additional top perspective views of automatic release 600 as an enhancement to door mechanism 500 of the above teachings. Not all the components of mechanism 600 from FIG. 19A-B are explicitly marked in FIG. 19C-D for clarity.

Automatic release 600 automatically opens door 110 of pod 100 when there is a power failure i.e. when power/electricity to pod 100 has been cut off, without requiring any user intervention. Now let us look at the operating or working principle of automatic release mechanism 600. According to the chief aspects, automatic release 600 is connected by a clutch pulling string 620 to string door mechanism 500 of FIG. 18 discussed above. In particular, clutch pulling string 620 is connected to the rack gear holder and pulling string connector 626 and clutch lever 580 of door mechanism 500.

Let us now review the operation of such an automatic release string door mechanism by taking advantage of FIG. 19 in conjunction with FIG. 18. When pod 100 is first energized and turned on, electronic control 602 detects the sensor in electromagnetic door latch 618 through the position of lock pin 622. This determines the condition of clutch DC motor 550 i.e. whether it is in an engaged or a disengaged condition. If the position of lock pin 622 is engaged to the electromagnetic door latch 618, this indicates that clutch DC motor 550 is engaged. If lock pin 622 is disengaged from electromagnetic door latch 618, this indicates that clutch DC motor or simply DC motor 550 is disengaged.

If DC motor 550 is disengaged, then electronic control 602 commands stepper motor 606 to pull clutch pulling string 620. The pulling is done via a combination of pinion gear 616 and rack gear 617. The pulling process continues until lock pin 622 has locked into electromagnetic door latch 618. This causes the clutch on DC motor 550 to be activated or engaged. If clutch DC motor 550 were already engaged, the pulling of clutch pulling string 620 is not carried out.

When power supply to pod 100 is suddenly cut off or is turned off, electronic control 602 triggers electromagnetic door latch 618. This causes the lock on lock pin 622 to open, while the clutch on DC motor 550 is deactivated/disengaged due to the pulling force of the combination of springs attached to clutch lever 580. If DC motor 550 were already deactivated/disengaged, the emergency door open process occurs as explained previously. Now, when the power supply to the pod is restored, the above process is repeated until clutch DC motor 550 is reactivated or re-engaged, and the normal door opening and closing processes explained above can be carried out. In summary, by releasing lock pin 622 in the case of a power failure, automatic release mechanism 600 disengages the clutch of its DC stepper motor 606 via clutch pulling string 620 that connects to the clutch lever of the door mechanism.

In view of the above teaching, a person skilled in the art will recognize that the apparatus and method of invention can be embodied in many different ways in addition to those described without departing from the principles of the invention. Therefore, the scope of the invention should be judged in view of the appended claims and their legal equivalents.

What is claimed is:

1. A pod chamber for delivering a multisensory experience to a user reclined inside said pod chamber, said pod chamber comprising:
(a) a first set of audio speakers behind the head of said user for delivering two coherent sound waves that produce an audio beat frequency;
(b) a second set of audio speakers on each side of the head of said user for delivering an audio performance in concert with said audio beat frequency;
(c) a stroboscopic light source for delivering a strobing pattern designed to be perceived by said user with eyes closed, said strobing pattern being in concert with said audio beat frequency and with said audio performance in order to produce a rapid eye movement in said user;
(d) a set of vibro-tactile transducers for delivering tactile vibrations to said user in concert with said audio beat frequency and with said audio performance; and
(e) a pulsed electromagnetic field generator unit for delivering electromagnetic waves to said user;
whereby said predetermined audio beat frequency, said audio performance, said strobing pattern, said tactile vibrations and said electromagnetic waves induce in said user a sense of participation in said multisensory experience.

2. The pod chamber of claim 1, wherein said audio beat frequency is between 6 Hertz and 100 Hertz.

3. The pod chamber of claim 2, wherein said audio beat frequency is 40 Hertz.

4. The pod chamber of claim 1, further comprising an olfactory aromatic diffuser for delivering aromas to said user in concert with said audio beat frequency and with said audio performance.

5. The pod chamber of claim 1, further comprising a roof screen above said user for delivering visuals in concert with said audio beat frequency and with said audio performance.

6. The pod chamber of claim 1, wherein said stroboscopic light source comprises light emitting diodes.

7. The pod chamber of claim 6, wherein said light emitting diodes produce light spanning the visible spectrum of natural sunlight.

8. The pod chamber of claim 1, wherein said set of vibro-tactile transducers is mounted below said user reclined inside said pod chamber.

9. The pod chamber of claim 8, wherein said set of vibro-tactile transducers comprise at least one transducer positioned under the lower spine, at least one transducer positioned under the gluteus maximus and at least one transducer positioned under the calves of said user reclined in said pod chamber.

10. The pod chamber of claim 1, further comprising a door for closing said pod to achieve a complete enclosure of said user reclined inside said pod.

11. The pod chamber of claim 10, wherein said door is controlled by an automatic release string door mechanism.

12. The pod chamber of claim 1, further comprising air ducts for circulating air to said user reclined inside said pod chamber.

13. A method of delivering a multisensory experience to a user reclined inside a pod chamber, said method comprising the steps of:
(a) delivering by a first set of audio speakers behind the head of said user, two coherent sound waves that produce an audio beat frequency;
(b) delivering by a second set of audio speakers on each side of the head of said user, an audio performance in concert with said audio beat frequency;
(c) delivering a strobing pattern by a stroboscopic light source designed to be perceived by said user with eyes closed, said strobing pattern being in concert with said audio beat frequency and with said audio performance in order to produce a rapid eye movement in said user;
(d) delivering tactile vibrations by a set of vibro-tactile transducers to said user in concert with said audio beat frequency and with said audio performance;
(e) delivering electromagnetic by pulsed waves a electromagnetic field generator unit to said user; and
(f) inducing in said user a sense of participation in said multisensory experience as a result of said audio beat frequency, said audio performance, said strobing pattern, said tactile vibrations and said electromagnetic waves.

14. The method of claim 13, wherein said audio beat frequency is between 6 Hertz and 100 Hertz.

15. The method of claim 14, wherein said audio beat frequency is 40 Hertz.

16. The method of claim 13 further delivering by an olfactory aromatic diffuser, aromas to said user in concert with said audio beat frequency and with said audio performance.

17. The method of claim 13 further delivering by a roof screen above said user, visuals in concert with said audio beat frequency and with said audio performance.

18. The pod chamber of claim 13, wherein said stroboscopic light source comprises light emitting diodes.

19. The pod chamber of claim 13, wherein said set of vibro-tactile transducers is mounted below said user reclined inside said pod chamber.

20. The method of claim 13 further providing a door for closing said pod chamber to achieve a complete enclosure of said user reclined inside said pod chamber.

21. The method of claim 20, wherein said door is controlled by a string door mechanism.

22. The method of claim 21, wherein said string door mechanism is an automatic release string door mechanism.

23. The method of claim 13 further circulating air to said user via air ducts arranged in said pod chamber.
